(19) United States
(12) Patent Application Publication
(10) Pub. No.: US 2004/0175765 A1
(43) Pub. Date: Sep. 9, 2004

(54) Cell-screening assay and composition

(76) Inventors: Sharat Singh, Los Altos Hills, CA (US); Po-Ying Chan-Hui, Oakland, CA (US)

Correspondence Address: ACLARA BIOSCIENCES, INC., 1288 PEAR AVENUE, MOUNTAIN VIEW, CA, 94043, US

(21) Appl. No.: 10/740,079

(22) Filed: Dec. 18, 2003

(30) Foreign Application Priority Data
Jul. 5, 2002 (WO) PCT/US02/21339

(51) Int. Cl. G01N 33/53; G01N 33/567

(52) U.S. Cl. 435/7.2

(57) ABSTRACT

The present invention discloses methods for multiplexed cellular assays. The methods can be used for a simultaneous quantitation of transcription levels from multiple promoters, multiple drugs, or for monitoring effects on multiple protein-protein interactions. These methods make use of reporter gene constructs that couple a single reporter enzyme coding sequence with one or more promoter regions, and a set of probes that are subject to degradation by the reporter enzyme, producing a set of reporters that can be separated from each other by differences in their mobilities. Multiplexed assays are achieved by combining a set of cell populations, wherein each population contains a distinct reporter gene construct and a distinct probe. Various treatments are then carried out on the mixture of cells. Treatments that induce transcription from specific promoters will cause synthesis of the reporter enzyme in that particular cell population, leading to degradation of the corresponding probe. The generation of specific reporters is determined by electrophoresis, or other means of measuring mobility, and is correlated with an effect of the treatment on transcription from a defined promoter.

### CROSS-REFERENCE TO RELATE APPLICATIONS AND PATENTS

This application claims priority to International application PCT/US02/21339 filed 05 Jul. 2002, U.S. provisional applications Ser. No. 60/304,296, filed 09 Jul. 2001 and U.S. provisional applications Ser. No. 60/325,292, filed 27 Sep. 2001, which applications are incorporated herein by reference in its entirety

### FIELD OF THE INVENTION

The present invention relates to methods and compositions for screening of the effects of a cellular treatment on protein expression in cells, and especially, methods and compositions suitable for multiplexed screening.

### REFERENCES

Buckholz, et al., J Mol Micro and Biotech (1999) 1(1): 135-40.

Deo, et al. J Anal Chem (2001) 369(3-4): 258-266.

Goetz, et al., J Biomol Screen (2000) 5(5): 377-384.

Hertzberg and Pope, Curr Opin Chem Biol (2000) 4(4): 445-451.

Holland, et al., Proc. Natl. Acad. Sci. USA (1991) 88:7276.

Lee, et al., Nucleic Acid Research (1993) 21(16): 3761.

Marino, et al., Electrophoresis (1996) 17:1499.

Meng, et al., Methods in Mol Biol (2001) 161: 255-268.

Olesen, et al. Methods in Enzymology (2000) 326: 175-202.

Silverman, et al., Curr Opin Chem Biol (1998) 2(3): 397-403.

Topcu, et al., Pharm Res (2000) 17(9): 1049-1055.

Walhout, et al., Yeast (2000) 17(2): 88-94.

White, Trends in Biotechnology (1996) 14(12): 478-483.

Zacharias, et al. Curr Opin Neurobiol (2000) 10(3): 416-421.

Zlokarnik, et al., Science (1998) 279: 84-88.

### BACKGROUND

With increasing emphasis on genetic-based drug therapies, there is growing interest in identifying compounds that are effective in controlling the level of transcription of proteins that may play a key role in pathological processes. Such compounds may act to interfere with protein expression indirectly, e.g., by inhibiting transcription activation pathways, such as certain kinase-dependent signaling reactions, or they may act directly, by interfering with the binding of a transcription factor on a promoter region of a gene.

Heretofore, high-throughput screening methods for detecting changes in transcriptional control of selected genes have been proposed. One general method involves transfecting cells, typically mammalian cells, with a genetic construct that includes a reporter gene under the control of the promoter (and in a broader sense, the upstream regulatory sequences) of the gene of interest. The reporter gene produced a protein, typically an exogenous protein that is either directly assayable, e.g., the luciferase gene, or one that can act enzymatically on a substrate to produce a detectable reporter compound. One such system has been described by Zlokarnik. The reporter gene in this system is a β-lactamase gene whose gene product, β-lactamase, can act on a chromogenic β-lactam substrate to produce a product that can be detected by a green-to-blue change in fluorescence.

Presently available methods for conducting reporter gene assays do not allow for a significant degree of multiplexing. To do so requires that multiple reporter genes be employed simultaneously in the same assay mixture, each gene product acting on a distinct detectable substrate. Such an approach is problematic in that it requires that a single assay condition be developed in which each reporter gene product is capable of showing detectable activity. However, only a limited number of reporter gene systems have been developed. Furthermore, as with most available homogeneous assay methods, rapid assay determinations generally rely upon light-emitting products, which can be multiplexed to a very limited degree by using dyes with different emission wavelengths. However, as in other applications, color multiplexing is very limited because of the spectral overlaps of available dyes.

A multiplexed assay format that would be advantageous for drug screening is one designed to assay the response of each of a plurality of genes, e.g., linked genes in a cell pathway, to a single test compound. In this format, each of a plurality of cells would be transfected with a construct composed of the promoter of one of the genes of interest, and a gene for a reporter gene, which would be the same for each different construct. However, in order to combine the separately transfected cell lines prior to drug screening, a means must be devised for providing a cell line-specific substrate for the reporter gene product, that in turn generates a cell line-specific product. This approach allows a large number of genes, and the transcriptional response of the genes, to be assayed under identical conditions, in a single assay.

Heretofore, such multiplexed transcription assays have not been practical, at least for more than a small degree of multiplexing that can be obtained using multiple reporter genes. The present invention is designed to provide multiplexed gene-transcription assay methods and compositions for use in such methods.

### SUMMARY OF THE INVENTION

The invention includes, in one aspect, a multiplexed assay for monitoring the level of transcription of one or more genes in response to one or more potential regulatory stimuli. The method includes placing transfected cells in each of a plurality of wells, where the cells in each well are transfected with a genetic construct comprising a selected promoter operatively linked to the coding sequence for an enzyme having a selected enzymatic activity. Exemplary enzymes include a β-lactamase, β-galactosidase, an esterase, a protease, or a nuclease. The probes may further include a transport moiety that facilitates transport of the probe into a cell, and a transport moiety linkage that is subject to cleavage within the cell to release the transport moiety and thereby inhibit probe transport of the probe out of the cell.

To each of the wells is added a probe selected from a set of probes. Each probe in the set is cleavable by the enzyme into a substrate moiety and an electrophoretic tag (e-tag) reporter having a detection group and a separation modifier that confers on the e-tag reporter, a unique electrophoretic mobility with respect to the e-tag reporters derived from the other probes in the set. The cells are incubated with the associated probes while exposing the cells to a potential regulatory stimulus.

Following the incubation step, the tags are obtained from the cells and electrophoretically separated. From the electrophoretic mobility and level of detection group of each separated e-tag reporter, the level of transcriptional response of each cell to the potential regulatory stimulus to which the cells were exposed is determined.

In one general embodiment, the incubating step may be carried out in separate wells, after which the cells are combined before obtaining the tags. The potential regulatory stimuli may include one or more test compounds, and the exposing step includes adding the test compound(s) to the individual cell-containing wells. For use in monitoring the dose response to a single test compound, the incubating step includes adding a different concentration of the test compound to each of a plurality of the wells. For use in monitoring the response to each of a plurality of different test compounds, the incubating step includes adding a different test compound to each of a plurality of the wells. For use in monitoring the response to a single test compound of each of a plurality of genes under the control of each of a plurality of different promoters, the cells in each of a plurality of wells are transfected with a different construct, and the incubating step includes adding the test compound to each of the plurality of the wells.

In another general embodiment, the incubating step is carried out in a single well containing a plurality of different cells, each containing a different probe. For use in monitoring the response to a single test compound of each of a plurality of genes under the control of each of a plurality of different promoters, the different cells in said well are each transfected with one of a plurality of different constructs, each comprising one of the promoters operatively linked to said coding sequence.

One exemplary probe, where the construct encodes a β-lactamase enzyme, has the form:

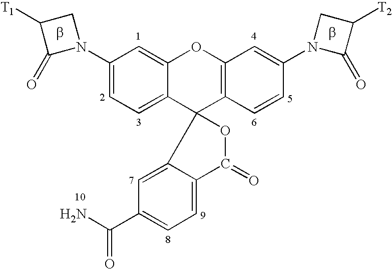

where exemplary positions of transport moieties are indicated at T₁ and T₂, the four-member rings indicated by β are β-lactam ring substrates for the enzyme, and a separation modifier is covalently carried at one of the numbered positions.

Another exemplary probe, where the construct encodes a β-lactamase enzyme, has the form:

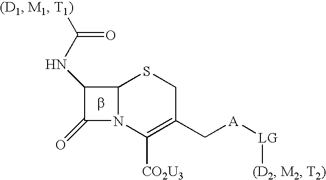

where exemplary positions of transport moieties are indicated at T₁ and T₂; for the detection groups, at D₁ and D₂; and for the separation modifiers, at M₁ and M₂. Cleavage of the lactam ring induces electron flow towards the acceptor, shown as A, causing cleavage of the probe by elimination of the leaving group, shown as LG. The two detection groups D₁ and D₂ may be capable of exhibiting efficient fluorescence resonance energy transfer; with cleavage of the β-lactam ring resulting in separation of the two fluorophores, thereby restoring fluorescent emission from the shorter wavelength fluorophor. One preferred probe of this type has the form:

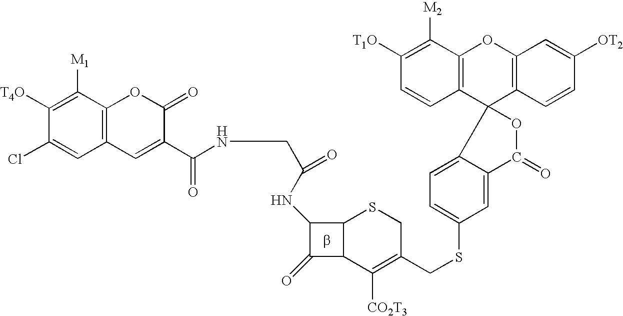

where exemplary positions of the transport moiety are shown as T₁, T₂, T₃ and T₄; the first detection group D₁ is a cephalosporin; the second detection group D₂ is a fluorescein; exemplary positions of the separation modifier are shown as M₁ and M₂; and the substrate is indicated as a four-member β-lactam ring, labeled β.

In one general embodiment, the detection group is a fluorescent moiety; in another, the detection group includes a catalytic moiety capable of catalyzing a detectable reaction.

The probes in the set may have the general form (D, Mⱼ)-S, where

(D, Mⱼ) is the detection group D linked to the separation modifier Mⱼ having a unique separation characteristic for each probe j in the set; S is a substrate for the enzyme; and the action of the enzyme on the probe produces an e-tag reporter of the form (D, M)-S′, where S′ is the residue of the substrate remaining with the e-tag reporter after reaction of S with the enzyme.

One general embodiment of the method is adapted for determining the extent of interaction of a first hybrid protein having a DNA-binding domain that binds to the selected promoter, and a first interaction domain; and a second hybrid protein having a transcriptional activation domain and a second interaction domain that is to be tested for interaction with the first interaction domain. The promoter is capable of activation by a polypeptide having a transcriptional activation domain when the transcriptional activation domain is in sufficient proximity to the gene. The cells contain the first and second hybrid proteins, and the determining step includes determining the extent of interaction of the two hybrid proteins by the level of e-tag reporter determined.

For use in screening a test compound for an effect on interaction between the first and second hybrid proteins, the method may further include adding the test compound to the cells and determining the amount of e-tag reporter produced, and from that, the extent to which the test compound has an effect on binding between the two hybrid proteins. Where the promoter is a repressible promoter, and the cells may further contain a repressor gene construct comprising an inducible promoter operatively linked to the coding sequence for a protein capable of binding to and repressing said repressible promoter.

For use in screening a test compound for an effect on interaction between the first and second hybrid proteins, the method may further include adding the test compound to the cells and determining the amount of e-tag reporter produced, and from that, the extent to which the test compound has an effect on binding between the two hybrid proteins.

In another embodiment, the method may be used to determine the extent of binding of a hybrid protein to a designated DNA sequence, where the hybrid protein has a transcriptional activation domain fused to a DNA-binding domain. The designated DNA sequence is operatively linked to the selected promoter, where (i) the promoter is capable of activation by a polypeptide having the transcriptional activation domain when the transcriptional activation domain is in sufficient proximity to the promoter, (ii) the cells contain the hybrid protein, and (iii) said determining includes determining the extent of binding of the hybrid protein to the designated DNA sequence by the level of e-tag reporter determined.

For use in screening a test compound for an effect on interaction between a hybrid protein and a designated DNA sequence, the method further includes adding the test compound to the cells after the mixing step, and comparing the determined amount of each separated reporter to the amount determined from cells that were not exposed to the test compound, thereby determining the effect of the compound on interaction between any of the hybrid protein and the designated DNA sequence.

In another aspect, the invention includes a probe composition for use in monitoring the level of transcription of an enzyme under the control of a plurality of different promoters. The composition includes a set of probes of the form: Tₓ-(D, Mⱼ)-S,

where Tₓ is one or more transport moieties that facilitate transport of the probe into a cell, and a transport moiety linkage that is cleavable within the cell to generate a probe by release of the transport moiety, said probe being inhibited from passage out of the cell; D is a detection group; Mⱼ is a separation modifier having a unique separation characteristic for each probe j in a set of n probes, where j=1 to n; S is a substrate for the enzyme, and the action of the enzyme on the probe produces an e-tag reporter of the form (D, Mⱼ)-S′, where S′ is the residue of the substrate remaining with the e-tag reporter after reaction of S with the enzyme.

In one embodiment, the detection moiety D is a fluorescent moiety; in another, the detection moiety includes a catalytic moiety capable of catalyzing a detectable reaction.

Where the substrate S is a substrate for the beta-lactamase enzyme, the probes may have one of the forms given above.

These and other objects and features of the present invention will become more fully apparent when the following detailed description of the invention is read in conjunction with the accompanying drawings.

### BRIEF DESCRIPTION OF THE FIGURES

FIGS. 1A-C illustrate one possible sequence of changes in the structure of a probe during the course of performing the method of the invention. FIG. 1A illustrates one embodiment of the structure of a probe introduced to a cell culture for transport into the cells. Once the probe is taken up by the cell, intracellular deacetylases will remove the acetyl groups, as shown in FIG. 1B, resulting in a probe structure that is too hydrophilic to efficiently pass across the cell membrane. FIG. 1C shows a reporter after recognition and action on the deacetylated probe by a reporter gene product.

FIGS. 2A and 2B illustrate an embodiment of the methods of the invention for monitoring the effects of a stimulus on transcription from a defined promoter. In this illustration, an external cellular stimulus causes expression of a reporter enzyme, resulting in action on a probe to release a reporter.

FIGS. 3A and 3B depict hypothetical electropherograms of the separation of probe and reporter molecules resulting from the cellular samples of FIGS. 2A and B. FIG. 3A shows a single peak corresponding to the unmodified probe. FIG. 3B shows a second peak arising as a result of enzyme action on the probe to yield a reporter, catalyzed by expression of the reporter enzyme.

FIG. 4 shows the structures of numerous exemplary reporters having different electrophoretic mobilities.

FIG. 5 is multiple electropherograms showing separation of individual reporters. The figure illustrates obtainable resolution of the reporters, which are identified by their ACLA numbers.

FIG. 6A illustrates an exemplary fluorescent lactam probe for use in the invention. Enzyme products of the probe resulting from successive esterase and lactamase cleavages are shown in FIGS. 6B and 6C.

FIG. 7 illustrates another exemplary fluorescent lactam probe for use in the invention.

FIG. 8A illustrates an exemplary fluorescent lactam probe according to the general structure shown in FIG. 7, in which the uncleaved probe is capable of exhibiting FRET. Enzyme products of the probe resulting from successive esterase and lactamase cleavages are shown in FIGS. 8B and 8C.

FIGS. 9A and 9B illustrate an embodiment of the methods of the invention for a multiplexed screen to monitor the effects of a stimulus on transcription from multiple promoters. FIG. 9A illustrates individual construction of separate cell lines prior to mixing to conduct the screen. FIG. 9B shows the effect of an external cellular stimulus on a mixture of three separate cell lines. Stimulation of transcription causes expression of a reporter enzyme in only one of the cell lines, resulting in cleavage of a probe specifically in the responsive cell line.

FIGS. 10A and 10B depict hypothetical electropherograms of the separation of molecules resulting from the cellular samples of FIGS. 9A and B. FIG. 10A shows three peaks corresponding to the three unmodified probes contained in the mixture of cells in the untreated state of FIG. 9A. FIG. 10B shows a second peak arising as a result of modification of the probe by the reporter enzyme in one of the three cell lines of the mixture.

FIGS. 11A and 11B illustrate an embodiment of the methods of the invention for studying protein-protein interactions based on the forward yeast two-hybrid approach. In this illustration, an external cellular stimulus leads to disruption of the interaction between two peptides, causing a loss in transcription of the reporter gene.

FIGS. 12A and 12B depict hypothetical electropherograms of the separation of probe and reporter molecules resulting from the cellular samples of FIGS. 11A and B. FIG. 12A shows two peaks corresponding to the unmodified probe and the released reporter. FIG. 12B shows loss of the reporter peak due to disrupted expression of the reporter gene.

FIGS. 13A-C illustrate assembly of transcriptional initiation complexes on copies of the same indicator gene. The complexes have common DNA-binding and RNA polymerase-binding domains, but each has different protein-protein interaction domains.

FIGS. 14A and 14B illustrate an embodiment of the methods of the invention in which an external cellular stimulus on a mixture of three separate cell lines causes disruption of the expression of a reporter enzyme in only one of the cell lines, resulting in loss of action on a probe specifically in the responsive cell line.

FIGS. 15A and 15B depict hypothetical electropherograms of the separation of probe and reporter molecules resulting from the cellular samples of FIGS. 14A and 14B. FIG. 15A shows three peaks corresponding to the three unmodified probes and three peaks corresponding to released reporters contained in the mixture of cells in the untreated state of FIG. 14A. FIG. 15B shows the loss of one of the reporter peaks due to disruption of expression of the reporter enzyme in one of the three cell lines of the mixture.

FIGS. 16A and 16B illustrate an embodiment of the invention utilizing a reverse two-hybrid method. In this illustration, an external cellular stimulus disrupts a protein-protein interaction, resulting in loss of expression of a repressor protein, and induction of expression of a reporter enzyme.

FIGS. 17A and 17B depict hypothetical electropherograms of the separation of probe and reporter molecules resulting from the cellular samples of FIGS. 16A and B. FIG. 17A shows one peak corresponding to the probe. FIG. 17B shows a second peak arising as a result of action on the probe by the reporter enzyme, to yield a reporter molecule.

### DETAILED DESCRIPTION OF THE INVENTION

I. Definitions

The terms below have the following meaning herein unless indicated otherwise.

In defining the term “probe,” it is useful to consider the functional components of the probe, as used in practicing the methods of the invention. The basic components of a probe, which also may be termed groups or moieties, include (1) a detection group, D, (2) a separation modifier, M, (3) a substrate, S, and optionally (4) one or more transport moieties, T. The function of a probe in the invention is to serve as a substrate for a designated enzymatic activity, wherein action of the designated enzyme on the probe results in modification of the probe to generate a corresponding reporter molecule. Such modifications generally comprise removal of a portion of the probe, or modification in the structure of the probe. The resulting reporter molecule minimally comprises the detection group and the separation modifier.

A “reporter,” or “reporter molecule” of the invention is the molecule generated by action of a designated enzyme on a probe that contains the detection group and separation modifier of the probe.

A “detection group,” abbreviated “D,” refers to a chemical group or moiety that is capable of being detected by a suitable detection system, or alternatively a means for generating a detection group. Means for generating a detection group may include either incorporation of a reactive group to form a bond with a detectable moiety, or the detection group may be a catalytic moiety capable of catalyzing synthesis of a detection group in an electrophoretic system.

One preferred detection group is a fluorescent moiety or other chromogenic moiety. The detection group will typically be common among a set or subset of different probes, but may also differ among probe subsets.

The “separation modifier,” abbreviated “M,” is a moiety that confers upon the probe or reporter molecule containing it, a “separation characteristic” that allows separation of each probe or reporter molecule from all other probes and reporters of a designated set. The type of separation characteristic used will typically be determined by the separation platform being employed for analysis of an assay.

One preferred mode of analysis is fluorescent detection of molecules that have been separated electrophoretically. In this mode, the separation characteristic will be a unique electrophoretic mobility, conferred upon the molecule predominantly by virtue of a unique charge-to-mass ratio (although other factors that affect electrophoretic mobility are contemplated, such as shape, hydrophobicity, molecular interaction, etc.) In general, the unique charge-to-mass ratio of a probe or reporter molecule is due to the chemical structure of the separation modifier, since the detection group and substrate residue (if any) will be common among a set of probes. However, it is recognized that the detection group can also make unique charge and/or mass contributions to the reporters and probes. For example, a set of probes may be made up of a first subset having a group of separation modifiers that impart unique electrophoretic mobilities to the subset when in combination with a first detection group having one defined charge and/or mass, and a second subset having the same group of mobility modifiers in combination with a second detection group with a different charge and/or mass, thus imparting electrophoretic mobilities that are unique among the combination of both subsets.

Another preferred mode of analysis is mass spectrometry, in which the separation characteristic will be a unique mass. In this embodiment, the unique mass of a probe or reporter will serve as the separation characteristic, and the separation modifiers within a set of probes will be any set of structures that vary in mass without significantly impacting the efficiency of detection of a given structure within a set.

Other separation platforms are also contemplated, including various types of chromatographic separations, conducted on, e.g., HPLC, including reverse phase, size exclusion, and affinity chromatography, among others.

The term “substrate,” abbreviated “S,” when used in the context of a probe or probe composition, refers to the part of the structure that is recognized by the designated reporter enzyme of a given assay, and is acted upon by the enzyme to make a product having a separation characteristic that is different from that of the unreacted probe. The action of the enzyme will most preferably be cleavage of the substrate, however modifications comprising additions, conversions, or other types of modification are also contemplated. Through the course of the detailed description of the invention, reference will usually be made to a cleavage reaction, although one of general skill in the art will appreciate the use of other types of enzymatic modifications for generation of reporter molecules that are distinguishable from the probe from which they were derived.

“Transport moiety,” abbreviated “T,” refers to a structural group within a probe that makes the probe more hydrophobic, and thereby able to be passively taken up by cells. In addition, the transport moieties will be linked to the probe by linkages that are readily cleaved by intracellular enzymes. Removal of the transport moieties converts a probe to a probe. The resulting probe is far more hydrophilic, and is thus inhibited from diffusing back out of the cell, or if so, from being taken up by another cell.

The term “promoter” as used herein, is intended to include any or all cis-acting sequences of a gene that control transcription of that gene. Thus, sequences intended to be included in the meaning of the word promoter include elements such as upstream regulatory sequences, upstream activating sequences, enhancers, operators, negative and positive regulatory elements, or any other sequence element that is involved in regulating transcription of a designated gene.

A “set”, “group”, or “library” of probes refers to a plurality of probes numbering typically at least five, and more typically 10-100 or more probes, wherein the plurality of probes have common substrate moieties and different separation characteristics. As used herein, the term “probe set” refers to a set of probes for use in detecting the level of reporter enzyme activity present in each or any of a plurality of known, selected cell lines. The separation modifiers in a set of probes are typically designated “M_{J}.” Where the set contains n members, each M_{J}, where J=from 1 to n, is a separation modifier having a unique separation characteristic among the probes in the set.

The term “multiplex” as applied to methods, assays, detection, analyses, and other processes, means that a given process is conducted for multiple samples, enzymes, targets, and/or molecules simultaneously in a single mixture. Various steps in the methods of the invention may be conducted individually or in a multiplexed fashion. A “multiplexed assay” means the activity of a common reporter enzyme in a plurality of different cell lines is measured simultaneously in a single sample, and/or the detection step conducted to analyze the results of an assay is carried out for multiple assays in a single separation.

“Level of transcription” refers to the amount of transcription of a selected gene under the control of a selected promoter. As determined in the present invention, the level of transcription of the gene is determined from the measured activity of an enzyme whose coding sequence is under the control of the promoter for that gene.

“Transfected cells” refers to cultured cells, typically mammalian cells, that have been transfected by an engineered gene construct comprising a selected promoter and the coding sequence for a reporter enzyme, typically an exogenous enzyme. The intracellular activity of the reporter enzyme can be assayed by its ability to act on a probe, thereby releasing a detectable reporter having a unique and defined characteristic that allows it to be separated from other reporters, e.g., by electrophoretic mobility, mass, or other separation properties.

“Regulatory stimulus” refers to a chemical, biochemical, or physical stimulus capable of altering (inducing or repressing) the expression level of a selected gene.

“Potential regulatory stimulus” refers to a compound being tested for its ability to induce or repress expression of a selected gene, through the gene promoter.

II. Overview of the Assay Methods

The present invention discloses methods for multiplexed cellular assays. The methods can be used for (i) simultaneous detection of transcriptional response of a given cell to multiple different regulatory stimuli, e.g., test compounds, or to different concentration of one or more test compounds, (ii) simultaneous detection of the transcriptional response of multiple cell lines to one or more regulatory stimuli, (iii) simultaneous quantitation of transcription levels from multiple promoters, or (iv) for monitoring effects on multiple protein-protein interactions.

A preferred embodiment of these methods makes use of one or more reporter gene constructs that couple a single reporter enzyme coding sequence with one or more of a plurality of different promoter regions, and a set of tagged probes, each having a unique separation characteristic, that are subject to modification by the reporter enzyme, producing a set of tagged reporters that can be separated from each other by differences between their separation characteristics, e.g. electrophoretic mobility, mass, shape, or other physical property.

In one general embodiment, the transfected cells are exposed to the regulatory stimuli, e.g., test compound(s), in individual assay wells. In this embodiment, the cells in each well, typically transfected with the same reporter construct, are exposed to one of a plurality of different probes, to allow uptake of the probe into the cells in each well. Thereafter, the different wells are exposed to one or more of a group of different regulatory stimuli, or differing concentrations of the same regulatory stimulus, being tested for the ability to regulate gene expression in the gene controlled by the construct promoter. Typically, the regulatory stimuli are test compounds, but other stimuli, such as heat, pH environment, concentration of external cations, or the presence of cells capable of interacting with the transfected cells may also be tested. The cells are then incubated under conditions that allow differential gene expression in response to the added stimulus. Following this, the released tag reporters are obtained from the cells, either before or after combining the cells from the different wells, and the combined tag reporters are separated. The level of transcriptional response of cells in each well can then be determined from the mobilities and amounts of the detected tag reporters, which are each uniquely associated with a given, known probe. In this embodiment, multiplexing is carried out at the detection step, but promoter constructs are assayed individually.

In another general embodiment, multiplexed assays are achieved by combining a set of cell populations, where each population contains a distinct probe and different cell type that has the potential for different response to one or more regulatory stimuli, e.g., test compounds. The different cell lines may be formed by transfecting different cell lines with the same promoter-enzyme coding sequence construct, or by transfecting the same (or different) cell line with each of a plurality of different constructs, each containing a different selected promoter (corresponding to the promoter of a gene of interest) and operably linked to the coding sequence of a desired assay enzyme, as discussed below. Cellular treatments that affect transcription from specific promoters will cause expression or repression of the gene for the reporter enzyme in that particular cell population, causing changes in the amount of degradation of the corresponding probe. The generation of specific reporters is determined by separation of the products of the cellular assay, and is correlated with an effect of the treatment on transcription from a defined reporter gene construct. In this embodiment, multiplexing occurs both at the cell-response step in the method, and at the detection step.

Another embodiment of the invention employs reporter gene constructs coupling one of a plurality of reporter enzyme coding sequences with one of a plurality of promoter regions. These genetic constructs are used in conjunction with a plurality of tagged probes, wherein each probe of the plurality comprises a substrate moiety for one of the plurality of reporter enzymes, and has a unique separation characteristic subject to modification by one of the plurality of reporter enzymes, thereby producing one of a plurality of tagged reporters that can be separated from each other by differences between their separation characteristics, as described above. A single cell line is transfected with a plurality of reporter gene constructs, each having a unique reporter enzyme, enabling a multiplexed determination of the effects of a cellular treatment on different genetic constructs in the same cell. This embodiment of the invention will require that the conditions of the assay be such that each and all of the reporter enzymes employed will be capable of showing enzymatic activity. Exemplary reporter enzymes for use in combination include, e.g., β-lactamase, β-galactosidase, luciferase, Agueorin, Green Fluorescent Protein (GFP), esterases, proteases, kinases, phosphatases, and nucleases. Where a given level of multiplexing N is obtained by employing multiple reporter enzymes, the level of multiplexing X that is obtained using a mixture of singly-transfected cell lines, each containing a distinct probe (as described in the preceding paragraph), can be increased to N(X),

Where multiple cell lines are transfected with one of a plurality of reporter enzyme coding sequences under transcriptional control of one of a plurality of promoter regions, the multiple lines can also be combined for a multiplexed assay. As with the embodiment above, conditions for the assay must allow each reporter enzyme employed to be active.

The present invention provides methods for multiplexed cellular assays. Any of the embodiments disclosed above can be used for a simultaneous quantitation of transcription levels from multiple cis-acting regulatory sequences (herein referred to generally as “Promoters”), or for monitoring effects on multiple protein-protein interactions that control transcription of multiple reporter genes.

These methods make use of reporter gene constructs that couple a single reporter enzyme coding sequence with different promoter regions, and a set of probes that are subject to modification by the reporter enzyme, producing a set of reporters that can be separated from each other by differences in their separation characteristics. These probes are derivatives of probes containing transport moieties, such as ester modifications, that provide for membrane permeabilization. Once within the cell, cellular enzymes remove these transport moieties, thereby trapping the probe within the cell. Multiplexed assays are achieved by generating independent cell lines that contain a distinct promoter region controlling expression of a common reporter gene, and a distinct probe, then combining a set of cell populations prior to cellular treatment. Those treatments that affect transcription from specific promoters will cause expression or repression of the gene for the reporter enzyme in that particular cell population, within the mixture of cell populations, causing changes in modification of the corresponding probe to produce the corresponding reporter. The generation of specific reporters is determined after separation of the reporter products of the reaction, and is correlated with an effect of the treatment on transcription from a defined promoter.

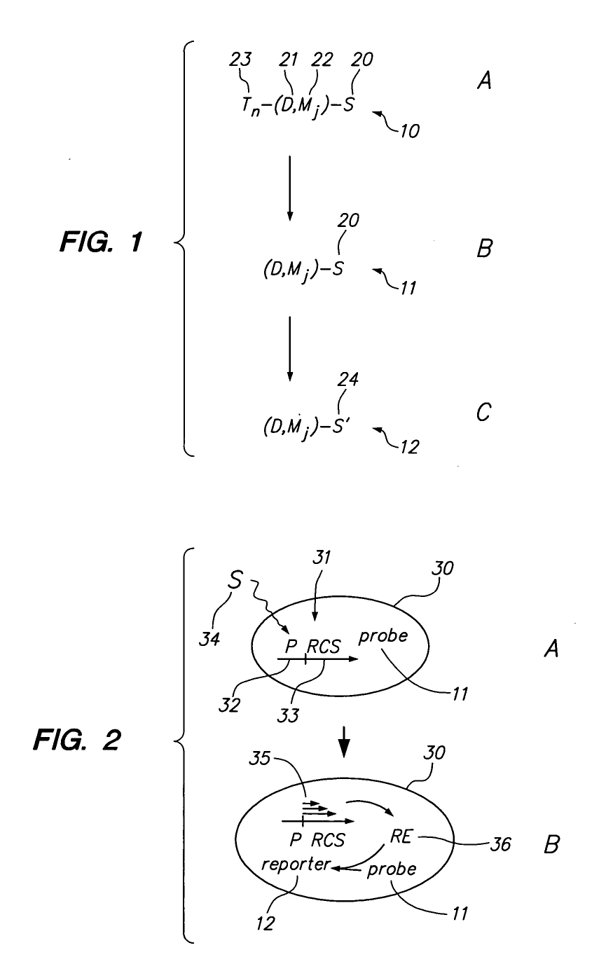

FIGS. 1A-1C illustrate a probe 10 and its transformations through the course of the method of the invention. The probe generally includes a substrate 20 that is linked to a tag that includes a detection group 21, such as a fluorescent reporter, and a separation modifier Mⱼ, indicated at 22, that imparts to the tag a selected separation characteristic, such as, e.g., charge-to-mass ratio, and thus a selected electrophoretic mobility. The detection group and separation modifier are show in parentheses to indicate that they may be represented in the structure in either order, or their functionalities may be embodied by a single moiety. The probe is a member of a set of n probes, where n=1 to J, each with a unique separation modifier Mⱼ that imparts a unique separation characteristic to the tag with respect to other tags in the probe set. All of the detection groups of a probe set may be the same, or multiple detection groups that can be simultaneously detected may be employed to further enhance the multiplexing capacity of the assay. Exemplary probes for a probe set useful in the invention will be described below.

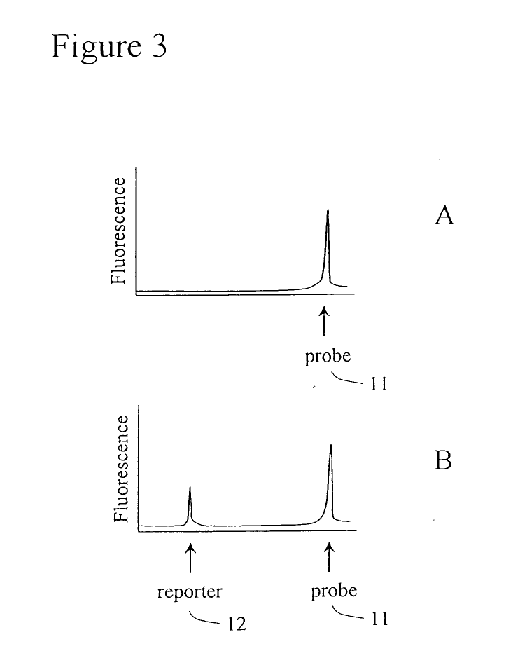

Also included in the probe are one or more transport moieties 23, generally ester groups, preferably acetyl groups, that facilitate transport of the probe across a cell membrane by rendering the probe nonpolar. The transport moiety is joined to the probe, normally through either the detection group or separation modifier, by a linkage, such as an ester linkage, that is cleavable by an intracellular enzyme, removing the transport moiety to generate the probe of the assay. Probe, indicated at 11 in FIG. 2B, is generated within the cell by removal of the transport moieties from the probe, rendering the resulting probe less hydrophobic, thereby inhibiting the rate of loss of the probe by transport out of the cell, and inhibiting any lost probe from being taken up by a second cell.

With modification of the probe, e.g., cleavage of the probe by the selected enzyme in the cell, the substrate or bulk of the substrate is released, as shown FIG. 1C, yielding an reporter 12 composed of the detection group 21, the separation modifier 22 and any residue S′ of the cleaved substrate, indicated as 24 in FIG. 1C. This reporter, by virtue of its unique separation modifier, can be electrophoretically separated from all other reporters derived from the set of probes in the multiplexed assay. The set of probes constitute one aspect of the invention, as exemplified by probe 10 in FIG. 1A, wherein each probe in the set comprises a different separation modifier. As a result of cleavage of the substrate moiety 20 by the reporter gene product, the reporter 12 will also have an electrophoretic mobility that is different from that of the probe itself 11.

FIGS. 2A and 2B illustrate cellular components that interact with the above-described probe, in a gene transcription assay carried out in accordance with the invention. The figures show a cell 30, typically a mammalian cell, which has been transfected with a genetic construct 31 containing a promoter of interest 32, indicated as P, and a reporter gene coding sequence 33 indicated as RCS, which is the coding sequence of a selected enzyme capable of cleaving probes in the probe set of the invention. In this embodiment, the promoter is one whose control and/or regulation, e.g., by test compounds, are to be studied in the method. Thus, the promoter is typically a mammalian promoter that is responsive to one or more trans-acting control elements capable of altering the level of transcription of the coding sequence operationally linked to that promoter. For the methods of the invention, the enzyme encoded by the reporter coding sequence is typically an exogenous enzyme, e.g., bacterial enzyme, such as β-lactamase, β-galactosidase, or other bacterial hydrolases, such as an esterase, protease or nuclease, in order to avoid the complications of background activity resulting from normal expression from the genome. In particular, it is desirable to employ as the reporter enzyme that is expressed, one that does not normally occur in the cells, or occurs only at low levels, or can be suppressed to low levels under selected growth conditions. Methods for selecting the genetic construct components, making the construct, and stably transfecting mammalian cells with the construct are described below.

In practicing the method, the transfected cells are mixed with a probe from the above probe set, such as probe 10 in FIG. 1A, which is taken up by the cells, and sequestered within the cells by cleavage of the transport moiety, thereby generating a probe, indicated as 11 in FIG. 2A. After the probe is taken up by the cell, the cells are exposed to a stimulus 34 indicated as S, which has the potential to impact the level of expression of the reporter gene, by acting at the level of promoter 32. The control may be either induction or repression, and the control exerted by the stimulus may be either indirect, e.g., by inhibiting or promoting the level of protein kinase action on an upstream control element, or direct, e.g., by inhibiting or promoting the binding of a regulatory factor, e.g., transcription factor, to the promoter. Typically the stimulus is a test compound whose ability to affect regulation of the selected gene is being assayed.

In the cell assay illustrated in FIGS. 2A and 2B, the stimulus applied is effective to induce transcription of the reporter gene under the control of the selected promoter. This, in turn, leads to increased levels of the reporter enzyme (RE), indicated at 36 in FIG. 2B. In turn, the enzyme interacts with probe 11, cleaving it into a reporter 12 and a substrate fragment.

By isolating the reporter from the cell, separating the reporter from other labeled molecules, and assaying the level of released reporter, the level of expression of the gene under the control of the selected promoter can be determined. In turn, by comparing the measured reporter level with that from cells that did not receive the stimulus, the effect of the stimulus on the transcriptional regulation of the reporter gene of interest can be determined. Typically, the reporter is isolated from the cells by cell lysis, and collection of a soluble cell fraction.

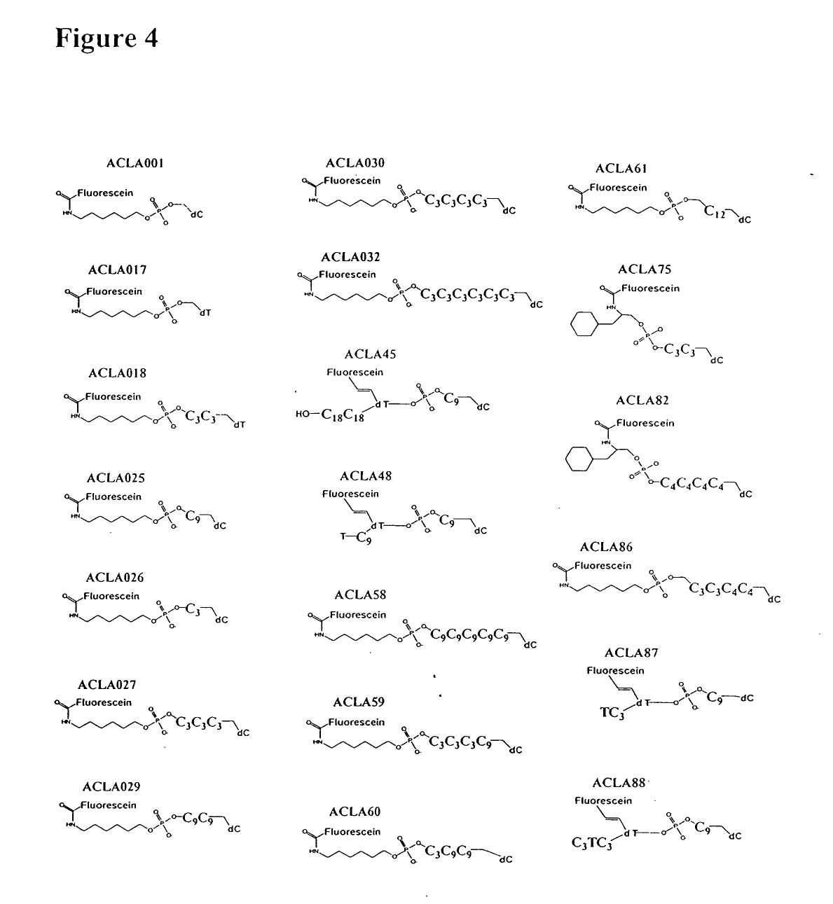

FIGS. 3A and 3B show electropherograms of detectable molecules isolated from cells before and after a stimulus that is effective to promote expression of the reporter enzyme. Before cellular treatment, and in the absence of expressed reporter enzyme, only intact probe will be detected, as shown in FIG. 3A. In cells in which a stimulus has been effective to promote gene expression, and thus expression of the reporter enzyme, the observed signal includes both intact probe and cleaved reporter molecules, as seen in FIG. 3B. By comparing levels of electrophoretic probes in the two figures, the ability of the stimulus to promote gene expression can be quantitated or semi-quantitated.

III. Probe Composition

The probes employed in the method are derivatives of probes used as reagents for the assay. The probes of the invention preferably comprise at least one transport moiety, such as one or more acetyl groups, that is attached to the probe through a bond that can be cleaved intracellularly, e.g., by a deacetylase. The transport moiety facilitates entry of the probes into the assay cells. Removal of the transport moiety likewise inhibits passage of the probe back out of the cells, or re-uptake of any lost probe by a second cell. The derivative probes of the invention have three important features. First, they will act as substrates for the reporter enzyme produced by the reporter gene construct. Second, each reporter released from its corresponding probe has a unique separation characteristic, e.g. charge-to-mass ratio, relative to that of other reporters from a set of probes (preferably combined with a relatively low mass, e.g., less than 1-5 kDal), that allows a plurality, e.g., 10 to 100 or more, different reporters, to be readily separated and uniquely identified according to each reporter's known separation characteristic, e.g., electrophoretic mobility. Finally, the resulting reporter will carry a detection group or means for generating a detection group. As discussed below, the detection group may be either a fluorescent or other chromogenic moiety, or a catalytic moiety capable of catalyzing synthesis of a detection group in an electrophoretic system.

In general, the probes in a set of n probes have the form Tₓ-(D, Mⱼ)-S, where Tₓ represents one or more transport moieties. After the probe is taken up by a cell, the transport moiety is removed by constitutive cellular enzymes, producing a probe of the form (D, Mⱼ)-S, where (D, Mⱼ) is the detection group D linked to a separation modifier Mⱼ, where j=1 to n for each probe in a set of n probes. S is the substrate moiety recognized by the reporter enzyme of the assay, wherein the action of the enzyme on the probe produces a reporter of the form (D, M)-S′, where S′ is the residue of S remaining with the reporter after reaction of S with the enzyme. D and M are shown in parentheses to indicate that there is no preferred order of these components in the probe or probe structure. Both T and S may be linked to the probe through either D or M.

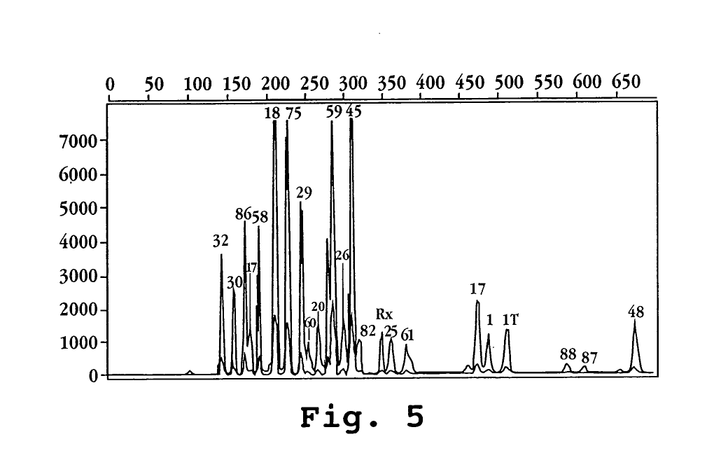

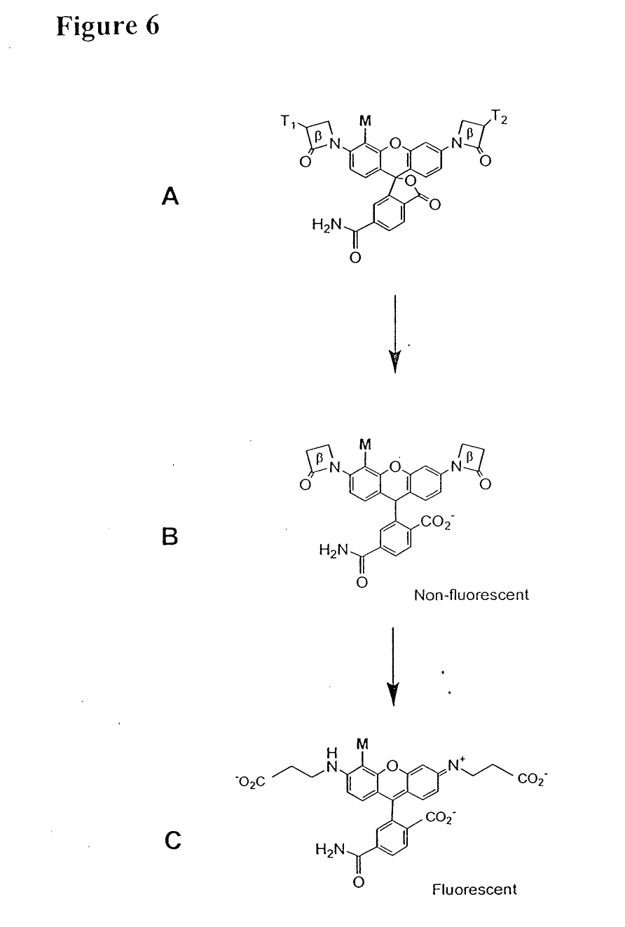

In one general embodiment, the detection group is a fluorescent moiety, such as fluorescein. FIG. 4 shows a number of suitable reporter structures derived from probe cleavage, each having a fluorescein detection group. These reporters comprise a tag moiety (D-Mⱼ) linked to an oligonucleotide through a phosphodiester linkage. As can be seen, the reporters have different separation modifiers, in addition to fluorescein and a single deoxynucleotide derived from the oligonucleotide probe that binds to target (shown as “dC” or “dT” in the figure). Each of these reporters has a unique charge/mass ratio that allows it to be separated from any of the other reporters on an electrophoretic platform. The ability to resolve each of the reporter structures electrophoretically is shown in FIG. 5.

The oligonucleotide probes containing the tag moieties shown in FIG. 4 would be suitable for use in an assay system in which the reporter enzyme (RE) is a nuclease. It will be appreciated that the (D-Mⱼ) groups may also be linked to a peptide substrate that can be cleaved by a selected peptidase, an ester linkage that allows cleavage with a selected esterase, a β-glucoside linkage that can be cleaved by a β-glucosidase, a β-lactam or β-lactam linkage that can be cleaved by a β-lactamase, or any other linkage or group that can be cleaved by a suitable cleaving enzyme, such as a hydrolase or phosphatase. The construction, structure, and electrophoretic properties of these and other suitable probes are described in co-owned U.S. Patent Application for “Tag Library Compounds. Compositions, Kits, and Methods of Use”, Ser. No. 09/698,846, filed Oct. 27, 2000, incorporated by reference and attached hereto.

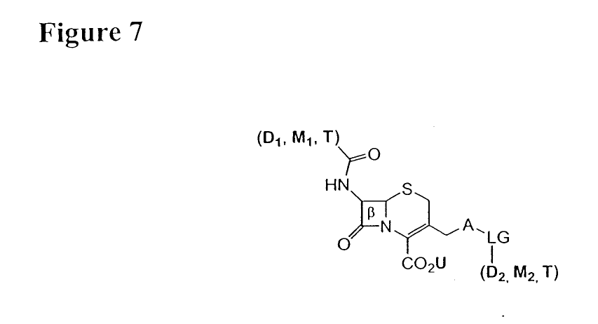

One preferred enzyme in the assay is a bacterial β-lactamase. FIG. 6A shows a β-lactam structure suitable for use as a probe in the invention, in which cleavage by β-lactamase either converts a leucodye to a fluorescent molecule, or causes a spectral shift in fluorescent emission. In structure 6A, the separation modifier is indicated as M, giving each resulting fluorescent reporter (FIG. 6C) a unique charge/mass ratio, and thus a unique electrophoretic mobility among a set of reporters. The figures show cleavage of the transport moieties, T, of the probe (6A) by an intracellular esterase to produce a probe (compound 6B), which serves as a substrate for a β-lactamase reporter enzyme. If the reporter enzyme is expressed in the cell, it will cleave the two lactam groups to produce the reporter shown in 6C. The compounds shown in FIGS. 6A and 6B are non-fluorescent leucodyes that are made fluorescent by cleavage of the lactam rings. In addition, cleavage of the lactam rings produces two carboxyl groups, thus significantly altering the charge/mass ratio of the compound, and rendering it easily separable from the non-cleaved precursor. This and other related compound structures suitable for use in the present invention are described in U.S. Pat. No. 6,031,094, which is incorporated herein by reference.

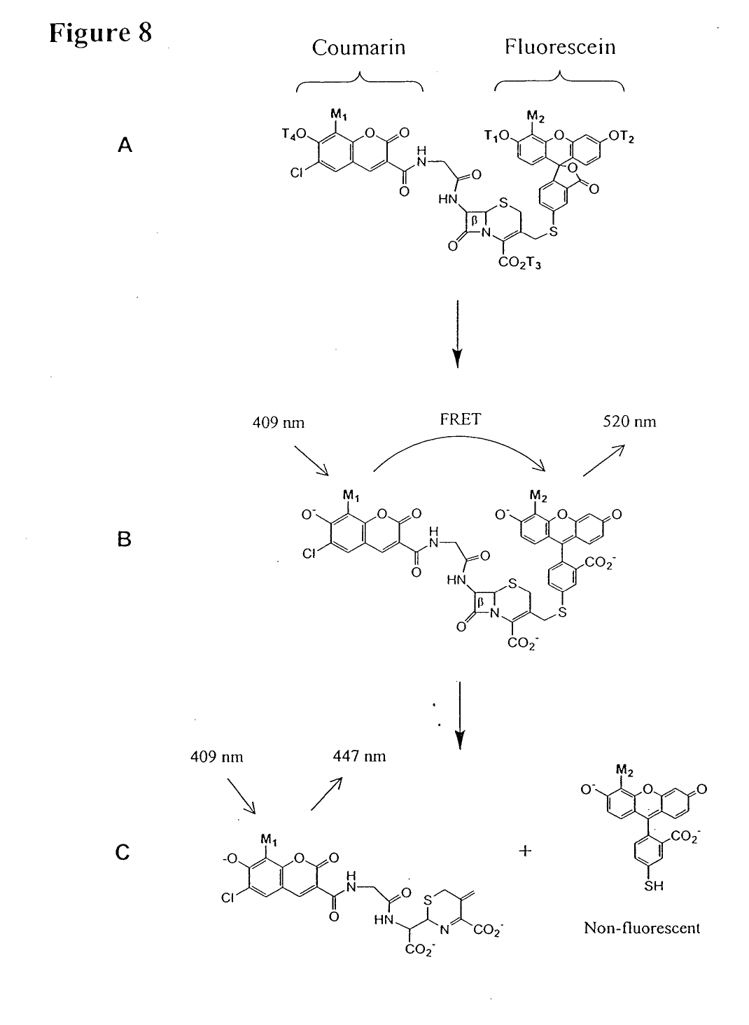

An exemplary structure of a second class of probes useful in practicing the invention, in which cleavage by β-lactamase causes elimination of a leaving group, is shown in FIG. 7. This structure comprises two fluorophores with overlapping spectra attached to the 7 and 3′ positions of a cephalosporin (Zlokarnik). The close proximity of the two fluorophores allows them to exhibit fluorescence resonance energy transfer, or FRET (J. R. Lakowicz, Principles of Fluorescence Spectroscopy, Plenum, N.Y., 1983.). FIG. 7A illustrates one embodiment of a probe in which 7-hydroxycoumarin is attached to the 7 position of cephalosporin, and serves as a donor fluorophor to fluorescein, which is attached to the 3′ position of the cephalosporin. Cleavage of the β-lactam ring of cephalosporin causes spontaneous elimination of the group attached to the 3′ position, in this case fluorescein, reestablishing fluorescence emission from the donor by disruption of FRET. The free thiol group remaining on the fluorescein as a leaving group completely quenches its fluorescence. The probe shown in FIG. 7A contains several transport moieties, which are removed by intracellular esterases to produce the compound shown in 7B, and preferably includes a separation modifier, M, attached to a non-quenched fluorescent product of β-lactamase cleavage. Action by a β-lactamase enzyme produces the two products shown in 7C. Additional embodiments of this class of probe structures include those reversing the position of attachment to cephalosporin of the donor and acceptor fluorophores.

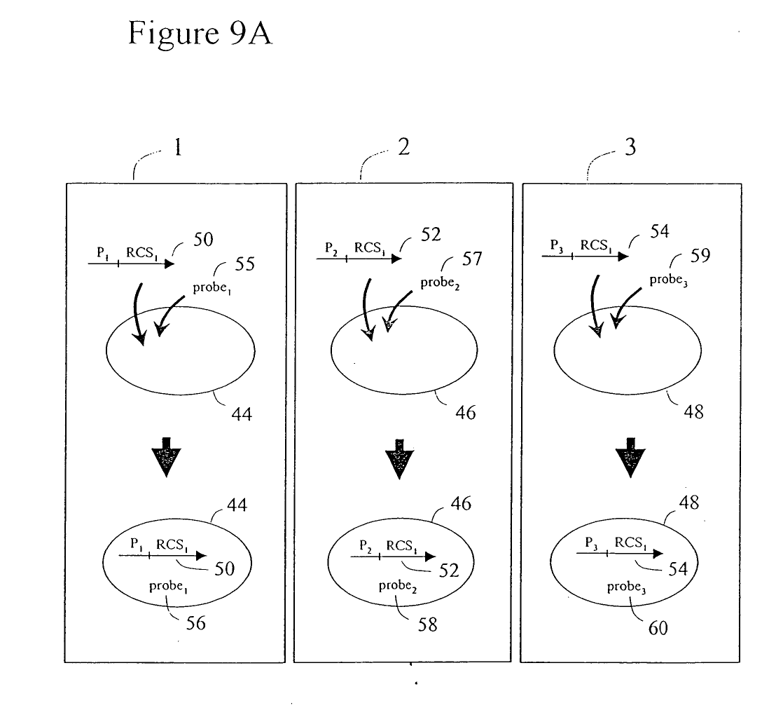

Probe structures with a leaving group removed by β-lactamase that do not make use of FRET also find use. One embodiment of a probe containing a single detectable moiety is shown in FIG. 8. This probe design comprises a fluorescent detection group D and separation modifier M attached to the 7 position of cephalosporin, and a non-fluorescent leaving group LG attached to the 3′ position of cephalosporin. As with the structure shown in FIG. 7B, cleavage of the probe by β-lactamase will lead to elimination of the leaving group, resulting in a change in the mobility of the resulting fluorescent reporter relative to the uncleaved probe.

Alternatively, the probes in a probe set may have, as their detection group, a moiety capable of catalyzing a localized detectable reaction, termed a catalytic group. In a general embodiment, the reporters produced by probe cleavage are separated electrophoretically, then combined with or exposed to reagents necessary for catalytic synthesis of a molecule that generates a detectable signal. For example, the catalytic group can be a photosensitive moiety capable of producing singlet oxygen in the presence of light. Singlet oxygen in turn reacts with a leucodye, oxidizing it to a fluorescent molecule. This embodiment may be accomplished by introducing substrates for the catalytic group into the electrophoretic medium, or by bringing the separated reporters into contact with the development reagents, e.g., in a T-junction within an electrophoretic system. In a second general embodiment, the released reporter molecules can serve as a required subunit in a multisubunit holoenzyme. Construction, structures, and properties of such catalytic electrophoretic tags, and systems for development detectable reactions with separated electrophoretic tag reporters, are described in co-owned U.S. patent application for “Methods and Reagents for Catalytic Multiplexed Assays ”, Ser. No. 09/293,821, filed May 26, 2001, incorporated by reference and attached hereto.

IV. Utilization of the Assay

Multiplexed Screening of Transcriptional Regulation

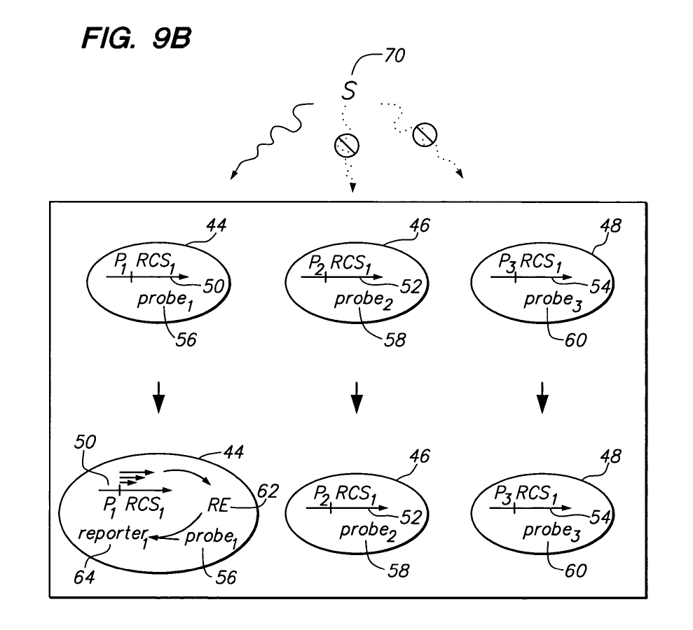

The foregoing is a description of the basic composition and operation of the components of the invention. In the following, the operation and components are applied to multiplexed screening methods. The first of these multiplexed cell assays, illustrated in FIGS. 9A and 9B, is designed to test the effect of a given stimulus, e.g., a test compound, on control of transcription from each of a plurality of different promoters, indicated in the figures as P₁, P₂, and P₃. FIG. 9A illustrates the processes conducted to prepare multiple cell cultures for combining in a multiplex assay. The figure shows three individual cell types, indicated at 44, 46, and 48. Prior to conducting a test with a stimulus, these separate cell cultures are transfected with a genetic construct that includes the promoter for one of the selected genes whose transcriptional response is being assayed, and a reporter coding sequence (RCS) for a selected reporter enzyme (RE), which is common to the set of constructs used in the assay. The genetic constructs in the three different cells are indicated at 50, 52, and 54 in the figures. In addition, each cell culture is incubated with a unique probe that can serve as a substrate for the RE. The probes, indicated at 55, 57, and 59, are taken up by the cells, and degraded intracellularly remove the transport moieties, thereby enhancing retention of the probes by the separate cell lines. The probes with the transport moieties removed are indicated at 56, 58, and 60, and are specific to the cell culture. A multiplexed assay may include a large number of such separately prepared cell cultures, e.g., 10-50 different samples, or more.

In the multiplexed assay illustrated in FIG. 9B, each of the cell cultures comprising the mixed sample is exposed to a stimulus 70, such as a test compound having potential regulatory effect on one or more of the promoters of interest. In the case illustrated, the stimulus induces transcription of the RCS under the control of promoter P₁, but not those under the control of promoters P₂ and P₃, with the result that the reporter enzyme RE is produced in cells of the first cell population only, as shown. To determine the assay results, the cells are lysed to obtain the probes and reporters from the mixture of cell lines, e.g., by isolating a soluble cell extract. The detectable molecules within the cell extract are then separated electrophoretically.

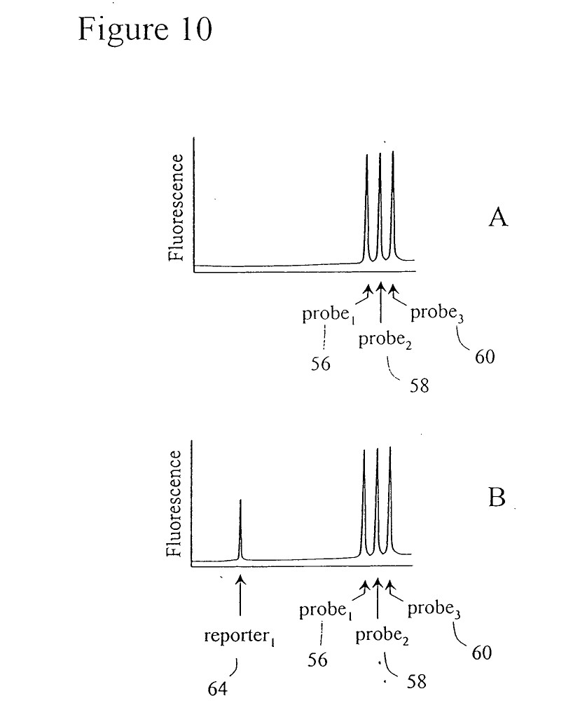

FIGS. 10A and 10B show electropherograms corresponding to an assay result before exposing the cells to stimulus (10A), and after exposure to a stimulus that induces transcription of the reporter gene under the control of promoter P₁, but not the genes under the control of promoters P₂ and P₃ (10B). Before exposure to the stimulus, the only peaks observed are those corresponding to uncleaved probes 56, 58, and 60. After exposure, a single cleaved reporter 64 is identified. The identity of the reporter is determined from the expected electrophoretic mobility of a reporter derived from the first probe 56, indicating that the stimulus acted only on the gene with promoter P₁. By integrating the peak, the amount of the cleaved probe is determined, and correlated with the level of induction of expression of the gene. Of course, in a multiplexed assay with many different promoters being tested, the electropherogram is likely to show a more complex pattern of probes and reporters. However, the known electrophoretic mobilities of these molecules, and their relative amounts, allows complex patterns of gene effects to be determined from the single electropherogram.

Essentially the same method can be employed to screen a plurality of test compounds in a single determination. This can be accomplished in two ways. Where the percentage of a compound library that is expected to elicit a change in gene expression is low, multiple compounds may be testing in a single cell mixture. Such a combinatorial approach is commonly used in compound library screening (REF), however the subject invention dramatically increases the amount of information obtained, as it allows a coupling of combinatorial screening with a plurality of gene expression targets.

A second method for screening a plurality of test compounds in a single determination can be employed where it is not advantageous to test multiple compounds in a single assay, such as in the case of an expectation of drug interactions. In this approach, after conducting single-compound assays on multiplexed cell populations, the cells from multiple independent assays are combined, and the probes and reporters are isolated and separated electrophoretically, as above.

Multiplexed Screening of Protein-Protein Interactions

The present method is also easily adapted to multiplexed methods for determining the interaction between two proteins, for example, in an assay to screen for a compound capable of inhibiting the interaction between two intracellular proteins. Here the assay format is a two-hybrid protein system, such as the yeast two-hybrid system, described in detail in for example, by Meng, Topcu, Walhout, and Buckholz. This assay is based on the reconstruction of a transcriptional activator from two hybrid proteins: a first hybrid protein containing a DNA-binding domain that binds to the selected promoter, and a first interaction domain; and a second hybrid protein having a second interaction domain to be tested for interaction with the first interaction domain, fused to a transcriptional activation domain. When the two proteins bind through their interaction domains, a functional transcriptional activator is produced, having both a DNA binding domain that binds to the promoter sequence, and an activation domain that is positioned to interact with RNA polymerase 11, promoting transcriptional activation of the gene of interest.

Two fundamental types of a two-hybrid screen include a “forward” two-hybrid system, in which interaction of the two hybrid proteins leads to expression of a reporter gene. A second type of two-hybrid screen is a “reverse” screen, in which interaction of the two hybrid proteins leads to expression of a repressor of transcription of a reporter gene. In this type of screen, a reporter gene signal is generated by disruption (rather than establishment) of the interaction between the two hybrid proteins.

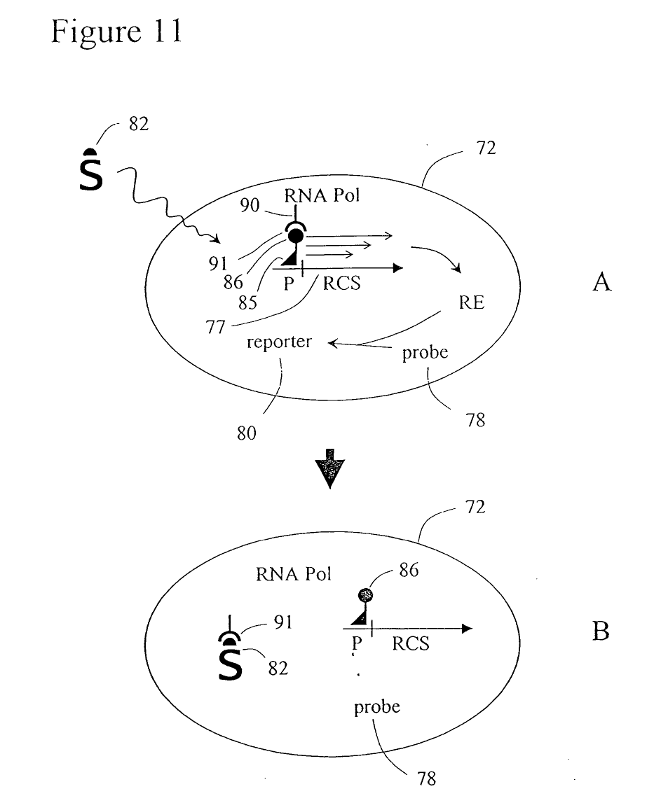

The basic components of a forward two-hybrid system, as they apply to the present invention, are illustrated in FIGS. 11A and 11B. The figures show a cell 72 that has been co-transfected with genetic constructs that produce the first and second hybrid proteins, typically by constitutive expression. The first hybrid protein includes a DNA binding domain, indicated at 85, and a first interaction domain of interest, indicated at 86. The second protein has a transcriptional activation domain, indicated at 90, that interacts with RNA polymerase II, and a second interaction domain, indicated at 91, to be tested for interaction with the first interaction domain 86. In the absence of a binding inhibitor, the two proteins interact to form a functional transcriptional activator that is effective to localize RNA polymerase II to the promoter and induce transcription of a reporter gene coding sequence RCS under the control of the promoter P.

The cells in the assay are also transfected with a genetic construct 77 of the type described above, having a selected promoter P controlling a reporter gene coding sequence encoding a reporter enzyme RE. As above, the cells are also contacted with a probe of the type described above, from a set of probes, allowing it to be taken up by the cells and modified by intracellular enzymes to produce a probe 78 that is retained within the cell.

In an untreated state, the two hybrid proteins interact, leading to the expression of the reporter enzyme RE, and conversion of probe 78 to a reporter 80, as shown in FIG. 11A. When exposed to an agent or stimulus 82 that inhibits or blocks interaction between the two hybrid proteins, as illustrated in FIG. 11B, then no functional transcription factor is produced, the gene for the reporter enzyme remains unexpressed, and probe 78 is uncleaved.

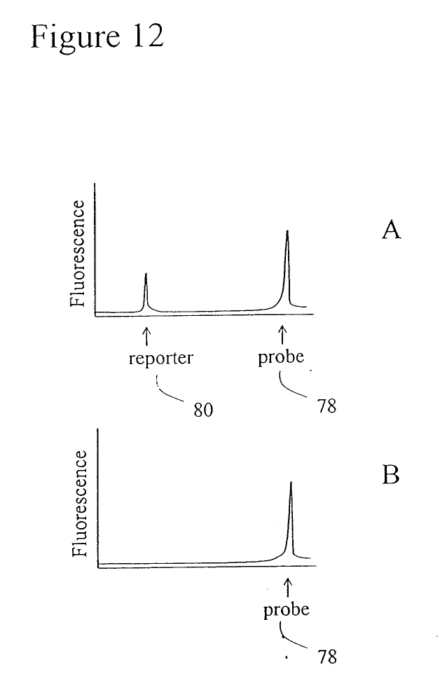

Electropherograms of cell lysate material containing probe and reporter molecules from the untreated and treated cells just described are shown in FIGS. 12A and 12B, respectively. The lack of detectable reporter in the treated-cell material indicates that the treatment or stimulus was effective to block interaction of the two hybrid proteins.

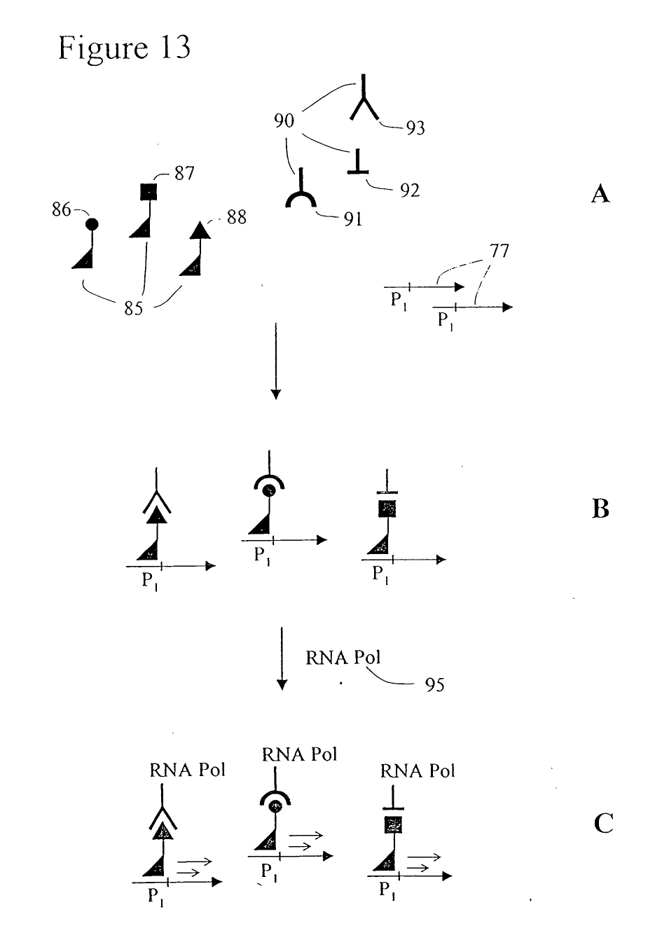

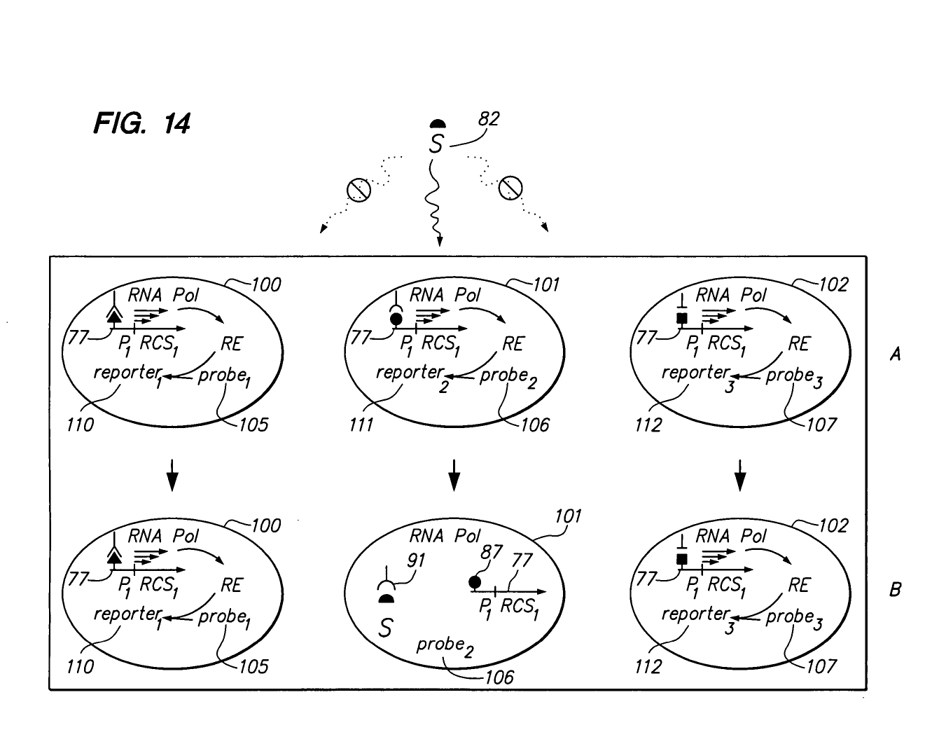

FIGS. 13 and 14 illustrate how a two-hybrid screening system can be multiplexed using the methods of the invention. FIG. 13 illustrates assembly of three different transcription initiation complexes on copies of the same indicator gene. FIG. 13A shows three different first hybrid proteins, each having the same DNA-binding domain, indicated at 85, that bind to the promoter sequence P₁ of a designated reporter gene 77, and different first interaction domains, indicated at 86, 87, and 88. Also shown are three different second hybrid proteins having a common transcriptional activation domain, indicated at 90, that interacts with RNA polymerase II, and three different second interaction domains, indicated at 91, 92 and 93, to be tested for interaction with the first interaction domains 86, 87 and 88. As shown in FIG. 13B, in the absence of a binding inhibitor, the specific pairs of hybrid proteins interact with each other to form a functional transcriptional activator that is effective to localize RNA polymerase 11 to the promoter and induce transcription of a gene under the control of the promoter P₁, shown in FIG. 13C.

Application of this multiplexed two-hybrid system to a multiplexed assay for the screening of a test compound on a set of protein-protein interactions is illustrated in FIG. 14. In this embodiment, a test compound S, indicated at 82, is tested for its ability to block or inhibit the binding of the two interacting moieties that together form a transcriptional activator in particular two-hybrid transcription factors. The figure shows three distinct cell lines, indicated at 100, 101, 102, generated separately, then combined in a single test mixture. Such an assay may include a large number of such cell lines, e.g., 10-50 different cell lines, or more. Prior to the screen, the cells in each cell line have been transfected with expression vectors for the two hybrid proteins, as well as a genetic construct 77 that includes a promoter P₁ of a selected gene whose transcriptional response is being measured, and the coding sequence RCS₁ for a selected reporter enzyme. A different selected probe is added to each cell line for transport into cells and conversion to a probe, indicated at 105, 106, and 107, prior to mixing the cell lines for the screen. As above, each probe will include a transport moiety that is cleaved once the probe enters the intracellular compartment, inhibiting its exit from the cell and subsequent re-uptake by a cell from a different line. The test agent 82 is then added to the mixed cell lines, and the cells are incubated under conditions, e.g., under growth and time conditions, that allow for detection in differences in transcription regulation. Following this, the cells are lysed and the probes and reporters extracted for analysis.

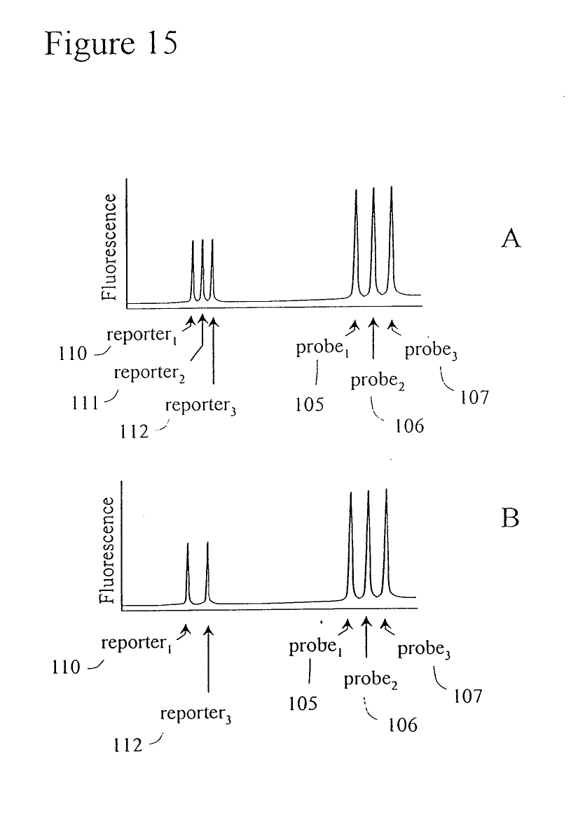

In the absence of added agent, the two hybrid proteins in each cell associate, forming a functional transcription factor, promoting expression of the reporter enzyme, which in turn leads to cleavage of the particular selected probe in each cell, thereby producing detectable reporters, indicated at 110, 111 and 112. An electropherogram of the detectable compounds would thus show each different probe, and each different reporter, as seen in FIG. 15A.

If a test compound added to a mixed cell sample is effective in blocking association of one of the hybrid protein pairs, as in the cell line 101 in the lower portion of FIG. 14, formation of a functional transcription factor for the P₁ promoter is inhibited, the RCS is not transcribed, and RE enzyme is not expressed of the cell. This is evidenced by the inability of those cells to cleave the intracellular probe to the corresponding reporter, or by reduced levels of the reporter. Similarly, if the added test agent has little or no effect on the binding of the two-hybrid interaction moieties, as indicated for the two cell lines 100 and 102 in FIG. 14, there is little or no effect on levels of expressed RE enzyme, and no change in corresponding levels of the probe and resulting reporter. An electropherogram of the probes and reporters from the mixed cell screen would then have the features indicated in FIG. 15B, showing “normal” levels of reporter₁ and reporter₃, but little or no reporter₂. The results obtained from the electropherograms in FIG. 15 can be used to identify specific protein-protein interactions that may be disrupted by a given agent.

The above embodiment illustrates use of the forward two-hybrid multiplexed system for testing the ability of a binding agent to block specific interacting moieties, by preparing a plurality of cell lines, each cell line having a designated pair of interaction moieties in the hybrid proteins and a designated probe. This embodiment may be used to screen for changes in hybrid protein interactions that are mediated by the test agent either directly, as shown in FIG. 14, or indirectly via, e.g., a signal transduction pathway. In other embodiments, the different cell lines will have one of a plurality of selected promoter sequences in the promoter/regulated enzyme construct and the first hybrid protein with the corresponding DNA-binding domain, and the interacting domains of the two hybrid proteins will be the same among the different cell lines. In this embodiment, the ability of a test agent to disrupt transcription from selected promoter regions can be monitored.

In contrast to the forward two-hybrid screening strategy illustrated in FIGS. 14 and 15, a reverse two-hybrid strategy will result in the gain of a signal (rather than loss) as a result of a change in a cell elicited by a cellular treatment. As such, this type of screen is advantageous for applications requiring high sensitivity, and is usually preferred for drug screening.

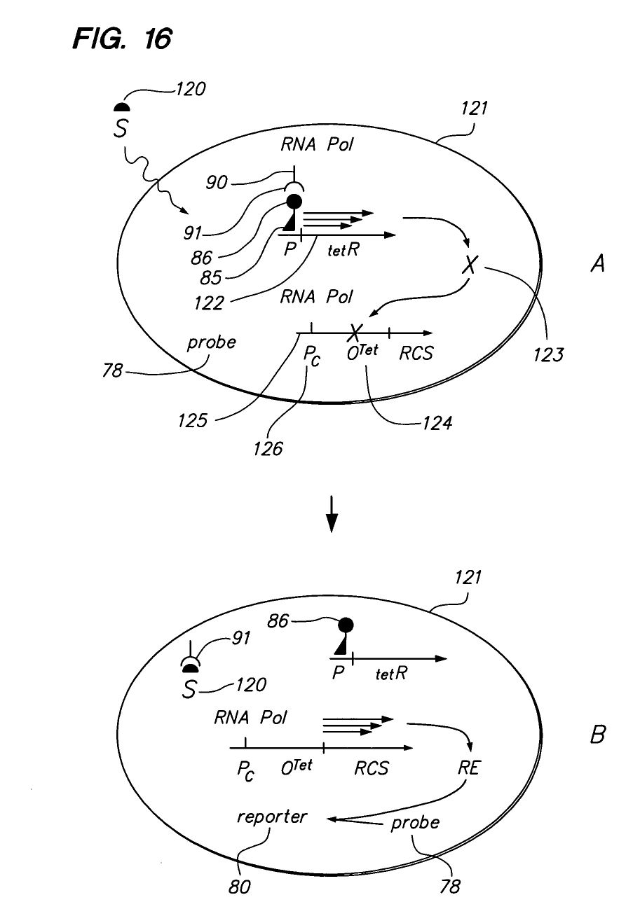

The basic components of a reverse two-hybrid system as they apply to the present invention, are illustrated in FIGS. 16A and 16B. As with the forward two-hybrid system described above, the figures show a cell 121 that has been co-transfected with genetic constructs that produce the first and second hybrid proteins, typically by constitutive expression. The first hybrid protein includes a DNA binding domain, indicated at 85, and a first interaction domain of interest, indicated at 86. The second protein has a transcriptional activation domain, indicated at 90, that interacts with RNA polymerase II, and a second interaction domain, indicated at 91, to be tested for interaction with the first interaction domain 86. In the absence of a binding inhibitor, the two proteins interact to form a functional transcriptional activator that is effective to bind to a designated site within a gene promoter P, thereby localizing RNA polymerase II to the promoter and inducing transcription of a gene under control of the promoter.

The reverse two-hybrid strategy differs from a forward two-hybrid approach, however, in that the gene 122 induced by interaction of the two hybrid proteins encodes a transcriptional repressor that acts in trans to block transcription of the reporter gene coding sequence RCS. In the embodiment shown in FIG. 16, the repressor gene is tetR, encoding the tetracycline gene repressor protein TetR, indicated at 123, which binds to an Operator sequence 124 normally present upstream of the tetracycline resistance gene. In this screen, a reporter gene 125 is constructed that positions the Tet Operator between a constitutive promoter 126 and a reporter gene coding sequence. The cells in the assay are transfected with the genetic constructs 122 and 125 of the type described above. As above, the cells are also contacted with a probe of the type described above, from a set of probes, allowing the probe to be taken up by the cells and modified by intracellular enzymes to produce a probe 78 that is retained within the cell.

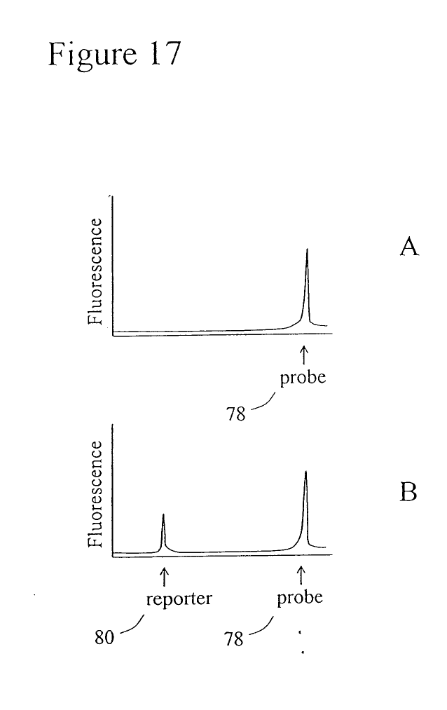

In an untreated state, as illustrated in FIG. 16A, the two hybrid proteins interact, leading to the expression of TetR, repressing expression of the reporter enzyme RE. In this state, the probe 78 will remain uncleaved. When exposed to an agent or stimulus 120 that inhibits or blocks interaction between the two hybrid proteins, illustrated in FIG. 16B, no repressor is produced, allowing transcription of the RE gene from a constitutive promoter, and conversion of probe 78 to a reporter 80. The type of results expected from this screen is shown in FIG. 17. Panel A shows a single peak arising from the uncleaved probe in an untreated cell. When the two-hybrid protein interaction is disrupted by a cellular treatment, reporter enzyme expression results in appearance of a second peak corresponding to the released reporter. As will be appreciated by one familiar with the methods described herein, this reverse two-hybrid approach is adapted to a multiplexed screening format as described above for the forward two-hybrid approach.

Multiplexed Screening of Protein-DNA Binding

A different type of hybrid protein approach called the one-hybrid system allows application of the methods of the invention to screens for DNA binding proteins. In this type of screen, a single hybrid protein is made, comprising a DNA binding domain of interest fused to a transcriptional activator domain. This type of screen is a more generalized approach to the methods described above for monitoring control of gene expression, except that interaction of any DNA-binding protein with any binding site is monitored. As with the forward and reverse two-hybrid strategies described above, either DNA binding or the disruption of DNA binding can be monitored.

V. Hybrid Protein Screening Systems

The cells employed in the method are typically mammalian cells that can be grown readily in culture. A variety of cell types, including cells derived from a variety of human normal and cancer tissues are available. The genetic reporter constructs used in the assay, each including the coding sequence for a reporter enzyme operatively connected to a selected promoter, is prepared according well-known recombinant methods. Promoter sequences of selected genes are available from a variety of sources, and sequences therefore can be obtained from standard sequence databases, such as GenBank, which can be accessed at www.ncbi.nlm.nih.gov/Genbank/. Likewise, coding sequences for a selected reporter enzyme, such as bacterial β-glucosidase, β-lactamase, a variety of nucleases, proteases, esterases, or phosphatases, or other hydrolytic enzymes, can be obtained from standard sequence sources, such as GenBank. Methods for constructing suitable expression vectors with a promoter operatively linked to the coding sequence for a heterologous protein, including enzymes, are detailed, for example, Zlorkarnik, and U.S. Pat. Nos. 6,245,512; 6,159,705; 6,153,381; 6,136,542; 6,117,639; 6,087,111; 6,063,578; 6,051,417; 6,048,693; 5,981,184; 5,969,210; 5,914,233; 5,853,985; 5,851,766; 5,830,728; 5,783,435; 5,723,291; 5,721,096; 5,643,726; 5,614,395; 5,585,232; 5,474,897; 5,445,941; 5,378,603; 5,346,812; and 4,761,367, all of which are concerned with expression constructs in assay cells. Similarly, these and other references provide ample detail for transfecting selected cells with the expression vectors.

As noted above, the probes to be delivered inside cells to serve as substrates for the reporter enzyme may contain transport moieties linked to the probes by a bond that is recognized by an intracellular enzyme, e.g., an intracellular deacetylase, such that the probes are trapped inside the cells following their transport into the cell and removal of the transport moiety. For a forward two-hybrid protein-protein interaction assay system, typical reporter constructs are exemplified by vectors such as those of the Hybrid Hunter™ system from Invitrogen, the MATCHMAKER LexA two-hybrid system from CLONTECH, and the DupLEX-A™ system from OriGene. Briefly, upstream of the reporter gene coding sequence is a Promoter sequence (P), which functions as a binding site for RNA polymerase and another sequence-specific DNA binding protein. The first hybrid protein fuses the first interacting domain to the DNA-binding domain of a sequence-specific DNA-binding protein, e.g. GAL4 (Clontech) or LexA (Invitrogen, OriGene), wherein the DNA-binding domain is capable of binding to the Promoter upstream of the reporter gene coding sequence. The second hybrid protein fuses the second interacting domain to a transcriptional activation domain, e.g. VP12 (Clontech) or B42 (Invitrogen, OriGene), which functions to interact with RNA polymerase to localize it to the gene in order to initiate transcription. The coding sequences for these two hybrid proteins are generally constructed in two separate expression plasmids.

For a forward one-hybrid protein-DNA interaction assay system, typical reporter constructs are exemplified by vectors such as those of the MATCHMAKER one-hybrid vector from CLONTECH and the Prey Plasmid from MoBiTec. Briefly, the reporter gene coding sequence is fused to an upstream regulatory sequence that promotes transcription of the reporter gene. The single hybrid protein used in this screen comprises the coding sequence for proteins being tested for binding to the upstream regulatory sequence fused to a transcriptional activation domain such as VP12 (Clontech) or B42 (MoBiTec). Exemplary reverse two-hybrid protein-protein interaction assay systems include the Split Hybrid system from Qbiogene. In this system, the LexA DNA-binding domain is fused to a first interacting domain to form a first hybrid protein. A second hybrid protein is formed from fusion of a second interacting domain to the VP16 transcriptional activator domain. The LexA binding site is inserted adjacent to the coding sequence for the tetracycline repressor (TetR), which in turn represses transcription of a designated reporter gene. When the interacting domains of the two hybrid proteins bind, TetR is made, causing repression of the reporter gene, and thereby repressing cleavage of a probe. When interaction between the two hybrid proteins is disrupted, repression of the reporter gene by TetR is removed, allowing positive screening for loss of interaction, observed as generation of a reporter from the corresponding probe.

VI. Cellular Screening

Cell lines containing the appropriate vector constructs are generated by transfection. For assays monitoring transcription directly, or for forward two-hybrid screens, cells may be transiently transfected for immediate screening. Long-term use of transfected cell lines requires that stable cell lines be generated by drug-resistance selection. Reverse hybrid assay systems require use of stable cell lines.

In carrying out a multiplexed assay, the transfected cell lines are individually incubated with a designated member of a plurality of probes to allow the probes to be taken up by the cells and conversion to probes that retained in the cells of a line. The amount of probe added is such as to yield easily detectable probe and reporter signal when the extracted probes and reporters are separated, e.g., electrophoretically. Following transport of probe into the cells, they may be washed to remove extracellular probe. Cell samples containing probes are preferably prepared immediately prior to performing a screen.

In the embodiment in which individual cell lines (typically the same cell line transfected with the same reporter construct) are incubated with different test compounds and/or with different concentrations of the same compound, prior to testing the cells in individual wells are exposed to different probes, to allow uptake of a different probe to the cells in each separate well. A different test compound or different concentration of the same compound (or for controls, no compound at all) are then added to each of the wells, and the cells are incubated under conditions that allow differential gene-transcription control. Typical incubation conditions involve incubation at 24-37° C. for several minutes to several hours, e.g., overnight. Thereafter, the tags released during the assay are obtained, either before or preferably after the cells are combined. The tags may be obtained, for example, by lysing the cells, and obtaining the tags in a soluble cell extract. The combined tags are then separated, and detected, e.g., fluorometrically, to determine the levels of transcription associated with each of the different test compounds or different concentrations of the same compound.

In a second general embodiment, the different cells, produced by transfecting different cell lines with the same gene construct, or the same cell line with different gene constructs, are first mixed with different probes, to generate different cell samples, each containing a distinct probe. The different cells, and the associated probes are then combined prior to conducting a gene-transcription assay. The mixed cell lines are placed in a common well, e.g., one of the wells in a microtitre plate, in order to test the transcription response of each of a plurality of promoters, or in the two-hybrid example, where the cells have different two-hybrid protein binding regions. The mixed cells are exposed to the assay stimulus, e.g., a test compound or one of a plurality of test compounds, and incubated in the presence of the stimulus for a period sufficient to observe changes in expression level of the reporter gene. For a transcriptional response, treatment with chemical compounds is generally performed overnight. Treatment with protein target candidates in the form of purified proteins or conditioned culture media involves pre-incubation at a certain dose in cell culture. The duration of treatment depends on the nature of the responses to be tested.

After the incubation/response step, the cells are washed and lysed to release intracellular probe and reporter compounds. Washing and lysing are carried out by conventional means, e.g., addition of a non-ionic surfactant. If desired the cell extract containing the probes and reporters may be concentrated, e.g., by lyophilization, or via a sample-stacking step during electrophoresis prior to electrophoretic separation. Alternatively, the probe substrate may include one member of a binding pair, such as an antigen, biotin, or the like, which allows the uncleaved probe to be captured on a solid-phase substrate coated with the other member of the binding pair, e.g., antibody or avidin, or may be reacted with a soluble form of the other binding pair, to produce a complex that does not compete with the reporters in the electrophoretic system employed. For example, the probe-binding member complex may have a charge opposite to that of the released reporter, at the pH of the electrophoretic medium employed.

The released reporters and, optionally, the soluble probes are then separated, e.g., electrophoretically, preferably using a capillary or microchannel electrophoretic system that allows separation and detection of small amounts of reporters. Such CE and microfluidic systems, and detector devices for quantitation of signals from separated bands, e.g., fluorescent signals, are well known. Electrophoretic conditions, e.g., separation medium, pH, and voltage levels are selected according to convention criteria to optimize separation.

The migration positions and relative levels of reporters, and optionally, probes, can be determined by analysis of electropherograms such as illustrated above in FIGS. 10 and 15. From this, the identity of the reporters and probes, i.e., the correspondence between given probes/reporters and given cell samples can be assigned, and the level of change in transcription of the selected gene(s) can be determined.

From the foregoing, it will be appreciated how various objects and features of the invention are met. In one embodiment, the method allows a large number of test compounds, and/or different concentrations of a single test compound, to be detected in a simple, multiplexed format that is both reliable and quantitative. As a result, levels of transcription in response to a large number of test compounds or test-compound concentrations can be identified and compared readily using simple electrophoretic separation and detection.

In addition, where different cells are being tested, e.g., cells transfected with constructs having different selected gene promoters, the method allows a large number of cellular responses to be quantitated using both a single cell-incubation format and a single detection format. The simultaneous screening and detection features simplify and speed up a screen, and provides for internal controls or standards for the measured levels of expression. The construction and structural features of the probes themselves, discussed below, permits electrophoretic separation of a large number of reporters in a single electrophoretic format, e.g., capillary electrophoresis, microfluidic device, conventional gel electrophoresis, HPLC, or mass spectrometry, thus permitting sample multiplexing of many samples, e.g., 10-100 samples, or more, at a time.

Although the invention has been described with respect to particular embodiments and materials, it will be appreciated that various changes and modifications may be made without departing from the invention.

It is claimed:

1. A multiplexed assay for monitoring the level of transcription of one or more genes in response to one or more potential regulatory stimuli, comprising:
placing transfected cells in each of a plurality of wells, where the cells in each well are transfected with a genetic construct comprising a selected promoter operatively linked to the coding sequence for an enzyme having a selected enzymatic activity;
adding to the cells in each well a probe selected from a set of probes, where each probe in the set is cleavable by the enzyme into a substrate moiety and an electrophoretic tag (e-tag) reporter having a detection group and a separation modifier that confers on the e-tag reporter, a unique electrophoretic mobility with respect to the e-tag reporters derived from the other probes in the set;
incubating the cells and associated probes while exposing the cells to a potential regulatory stimulus;
obtaining the tags from the cells;
electrophoretically separating the combined tags; and
determining, from the electrophoretic mobility and level of detection group of each separated e-tag reporter, the level of transcriptional response of each cell to the potential regulatory stimulus to which the cells were exposed.

2. The method of claim 1, wherein said incubating is carried out in separate wells, and said obtaining includes combining the cells from separate wells, and obtaining said tags from the combined cells.

3. The method of claim 2, wherein the potential regulatory stimuli include one or more test compounds, and said exposing includes adding the test compound(s) to the individual cell-containing wells.

4. The method of claim 3, for monitoring the dose response to a single test compound, with respect to the level of transcription of a selected gene under the control of a selected promoter, wherein the cells in each well are transfected with a construct comprising said promoter operatively linked to said coding sequence, and said incubating includes adding a different concentration of the test compound to each of a plurality of the wells.

5. The method of claim 3, for monitoring the response to each of a plurality of different test compounds, with respect to the level of transcription of a selected gene under the control of a selected promoter, wherein the cells in each well are transfected with a construct comprising said promoter operatively linked to said coding sequence, and said incubating includes adding a different test compound to each of a plurality of the wells.

6. The method of claim 1, for monitoring the response to a single test compound of each of a plurality of genes under the control of each of a plurality of different promoters, with respect to the level of transcription of the genes, wherein the cells in each of a plurality of wells are transfected with a different construct comprising one of said promoters operatively linked to said coding sequence, and said incubating includes adding the test compound to each of the plurality of the wells.

7. The method of claim 1, wherein said incubating is carried out in a single well containing a plurality of different cells, each containing a different probe, and said obtaining includes obtaining released tags from the cells in said well.

8. The method of claim 7, for monitoring the response to a single test compound of each of a plurality of genes under the control of each of a plurality of different promoters, with respect to the level of transcription of the genes, wherein the different cells in said well are each transfected with one of a plurality of different constructs, each comprising one of said promoters operatively linked to said coding sequence.

9. The method of claim 1, wherein the enzyme is selected from the group consisting of β-lactamase, β-galactosidase, an esterase, a protease, and a nuclease.

10. The method of claim 1, wherein each probe in the set further comprises a transport moiety that facilitates transport of the probe into a cell, and a transport moiety linkage that is subject to cleavage within the cell to release the transport moiety and thereby inhibit probe transport of the probe out of the cell.

11. The method of claim 10, wherein said enzyme is β-lactamase, and one or more of the probes of the set has the form: 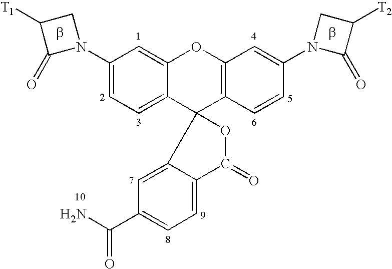
where exemplary positions of transport moieties are shown as T₁ and T₂;
a substrate for the enzyme, S, is exemplified as a four-member β-lactam ring, labeled β;
the main part of the structure comprises a fluorescein derivative, wherein various positions are numbered that are subject to chemical modification; and
a separation modifier, M, (not shown) is present at one of the numbered positions.

12. The method of claim 10, wherein said enzyme is β-lactamase, and one or more of the probes of the set has the form: 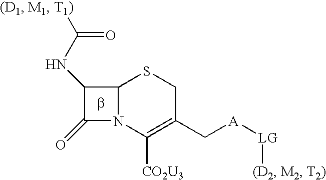
where exemplary positions of transport moieties are shown as T₁, T₂ and T₃;
exemplary positions of detection groups are shown as D₁ and D₂;
exemplary positions of separation modifiers are shown as M₁ and M₂; and
a substrate for the enzyme, S, is exemplified as a four-member β-lactam ring, labeled β, wherein cleavage of the ring induces electron flow towards the acceptor, shown as A, causing cleavage of the probe by elimination of the leaving group, shown as LG.

13. The method of claim 12, wherein
the probes comprise fluorophores at positions D₁ and D₂;
the two fluorophores are capable of exhibiting efficient fluorescence resonance energy transfer; and
cleavage of the β-lactam ring results in separation of the two fluorophores, thereby restoring fluorescent emission from the shorter wavelength fluorophor.

14. The method of claim 13, wherein one or more of the probes of the set have the form: 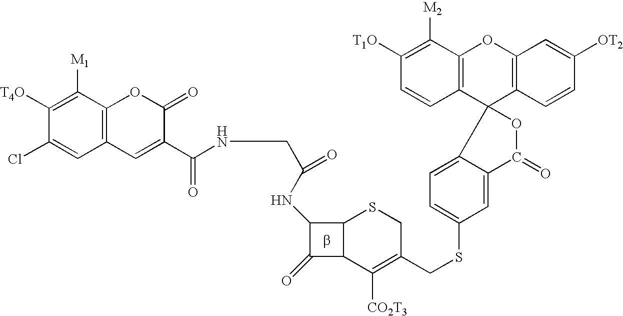
where exemplary positions of the transport moiety are shown as T₁, T₂, T₃ and T₄;
the first detection group D₁ is exemplified as a cephalosporin;
the second detection group D₂ is exemplified as a fluorescein;
exemplary positions of the separation modifier are shown as M₁ and M₂; and
the substrate is exemplified as a four-member β-lactam ring, labeled β.

15. The method of claim 1, wherein said detection group is a fluorescent moiety.

16. The method of claim 1, wherein said detection group includes a catalytic moiety capable of catalyzing a detectable reaction.

17. The method of claim 1, wherein the probes in the set have the form (D, Mⱼ)-S, where
(D, Mⱼ) is the detection group D linked to the separation modifier Mⱼ having a unique separation characteristic for each probe j in the set;
S is a substrate for the enzyme; and
the action of the enzyme on the probe produces an e-tag reporter of the form (D, M)-S′, where S′ is the residue of the substrate remaining with the e-tag reporter after reaction of S with the enzyme.

18. The method of claim 1, for determining the extent of interaction of a first hybrid protein having a DNA-binding domain that binds to the selected promoter, and a first interaction domain; and a second hybrid protein having a transcriptional activation domain and a second interaction domain that is to be tested for interaction with the first interaction domain; where (i) said promoter is capable of activation by a polypeptide having a transcriptional activation domain when the transcriptional activation domain is in sufficient proximity to the gene, (ii) cell contains said first and second hybrid proteins, and (iii) said determining includes determining the extent of interaction of the two hybrid proteins by the level of e-tag reporter determined.

19. The method of claim 18, for use in screening a test compound for an effect on interaction between the first and second hybrid proteins, which further includes adding the test compound to the cells and determining the amount of e-tag reporter produced, and from that, the extent to which the test compound has an effect on binding between the two hybrid proteins.

20. The method of claim 18, wherein the promoter is a repressible promoter, and the cells further contain a repressor gene construct comprising an inducible promoter operatively linked to the coding sequence for a protein capable of binding to and repressing said repressible promoter.

21. The method of claim 20, for use in screening a test compound for an effect on interaction between the first and second hybrid proteins, which further includes adding the test compound to the cells and determining the amount of e-tag reporter produced, and from that, the extent to which the test compound has an effect on binding between the two hybrid proteins.

22. The method of claim 1, for determining the extent of binding of a hybrid protein to a designated DNA sequence, said hybrid protein having a transcriptional activation domain fused to a DNA-binding domain, said designated DNA sequence being operatively linked to the selected promoter, where (i) the promoter is capable of activation by a polypeptide having the transcriptional activation domain when the transcriptional activation domain is in sufficient proximity to the promoter, (ii) the cells contain the hybrid protein, and (iii) said determining includes determining the extent of binding of the hybrid protein to the designated DNA sequence by the level of e-tag reporter determined.

23. The method of 22, for use in screening a test compound for an effect on interaction between a hybrid protein and a designated DNA sequence, which further includes adding the test compound to the cells after the mixing step, and comparing the determined amount of each separated reporter to the amount determined from cells that were not exposed to the test compound, thereby determining the effect of the compound on interaction between any of the hybrid protein and the designated DNA sequence.

24. A probe composition for use in monitoring the level of transcription of an enzyme under the control of a plurality of different promoters, comprising a set of probes of the form: T-(D, Mⱼ)-S, where
T is an transport moiety that facilitates transport of the probe into a cell, and an transport moiety linkage that is cleavable within the cell to generate a probe by release of the transport moiety, said probe being inhibited from passage out of the cell;
D is a detection group;
Mⱼ is a separation modifier having a unique separation characteristic for each probe j in the set;
S is a substrate for the enzyme, and
the action of the enzyme on the probe produces an e-tag reporter of the form (D, Mⱼ)-S′, where S′ is the residue of the substrate remaining with the e-tag reporter after reaction of S with the enzyme.

25. The composition of claim 24, wherein D is a fluorescent moiety.

26. The composition of claim 24, wherein said detection group includes a catalytic moiety capable of catalyzing a detectable reaction.

27. The composition of claim 24, wherein S is a substrate for the beta-lactamase enzyme.

28. The composition of claim 27, wherein one or more of the probes of the set has the form: 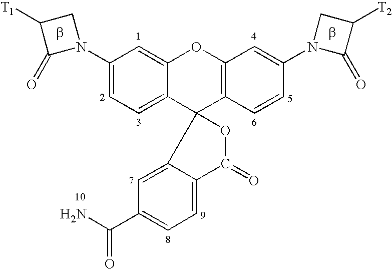
where exemplary positions of transport moieties are shown as T₁ and T₂;
a substrate for the enzyme, S, is exemplified as a four-member β-lactam ring, labeled β;
the main part of the structure comprises a fluorescein derivative, wherein various positions are numbered that are subject to chemical modification; and
a separation modifier Mⱼ (not shown) is present at one of the numbered positions.

29. The composition of claim 27, wherein one or more of the probes of the set have the form: 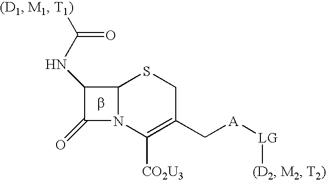
where exemplary positions of transport moieties are shown as T₁, T₂ and T₃;
exemplary positions of detection groups are shown as D₁ and D₂;
exemplary positions of separation modifiers are shown as M₁ and M₂; and
a substrate for the enzyme, S, is exemplified as a four-member β-lactam ring, labeled β, wherein cleavage of the ring induces electron flow towards the acceptor, shown as A, causing cleavage of the probe by elimination of the leaving group, shown as LG.

30. The composition of claim 29, wherein
the probes comprise fluorophores at positions D₁ and D₂;
the two fluorophores are capable of exhibiting efficient fluorescence resonance energy transfer; and
cleavage of the β-lactam ring results in separation of the two fluorophores, thereby restoring fluorescent emission from the shorter wavelength fluorophor.

31. The composition of claim 30, wherein one or more of the probes of the set have the form: 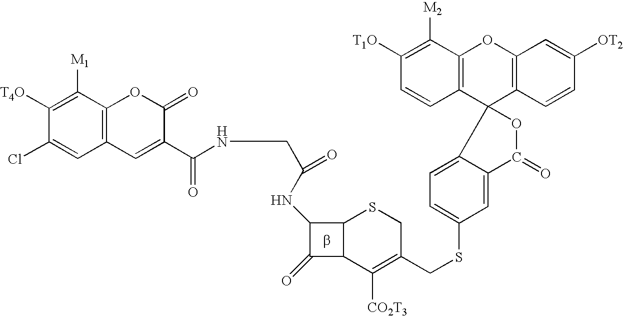
where exemplary positions of the transport moiety are shown as T₁, T₂, T₃ and T₄;
the first detection group D₁ is exemplified as a cephalosporin;
the second detection group D₂ is exemplified as a fluorescein;
exemplary positions of the separation modifiers are shown as M₁ and M₂; and
the substrate is exemplified as a four-member β-lactam ring, labeled β.
